(19) United States
(12) Patent Application Publication
(10) Pub. No.: US 2023/0416210 A1
(43) Pub. Date: Dec. 28, 2023

(54) METHODS OF MAKING A PPAR-DELTA AGONIST

(71) Applicant: Reneo Pharmaceuticals, Inc., Irvine, CA (US)

(72) Inventor: Michael Trevelyan WILLIAMS, Irvine, CA (US)

(21) Appl. No.: 18/253,023

(22) PCT Filed: Nov. 19, 2021
(86) PCT No.: PCT/US2021/060093

§ 371 Date: May 15, 2023

Related U.S. Application Data
(60) Provisional application No. 63/118,435, filed on Nov. 25, 2020.

(51) Int. Cl. C07D 265/30; B01J 23/44

(52) U.S. Cl. CPC C07D 265/30; B01J 23/44

(57) ABSTRACT

Described herein are methods of making the PPARδ agonist compound (E)-2-(4-((3-(4-fluorophenyl)-3-(4-(3-morpholinoprop-1-yn-1-yl)phenyl)allyl)oxy)-2-methylphenoxy)acetic acid, including salt forms thereof.

### CROSS-REFERENCE TO RELATED APPLICATIONS

This application claims benefit of U.S. Provisional Patent Application No. 63/118,435, filed on Nov. 25, 2020, which is incorporated herein by reference in its entirety.

### FIELD OF THE INVENTION

Described herein are methods of making a peroxisome proliferator-activated receptor delta (PPARδ) agonist compound.

### BACKGROUND OF THE INVENTION

PPARδ, a member of the nuclear regulatory superfamily of ligand-activating transcriptional regulators, is expressed throughout the body. PPARδ agonists induce genes related to fatty acid oxidation and mitochondrial biogenesis. PPARδ also has anti-inflammatory properties.

### SUMMARY OF THE INVENTION

Described herein are methods of making the PPARδ agonist (E)-2-(4-((3-(4-Fluorophenyl)-3-(4-(3-morpholinoprop-1-yn-1-yl)phenyl)allyl)oxy)-2-methylphenoxy)acetic acid (Compound I), and pharmaceutically acceptable salts thereof (e.g. the sodium salt).

In one aspect, described herein is a process for the preparation of the Compound II:

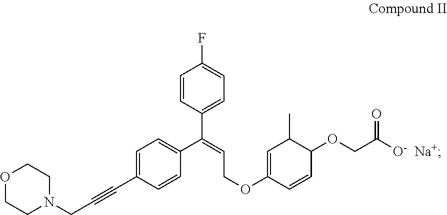

comprising:
(1) reacting Compound 3, or a salt thereof:

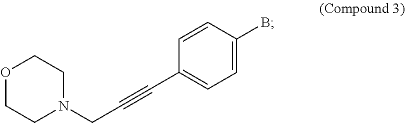

wherein B is a boronic acid, boronate ester, or trifluoroborate;
with Compound 4:

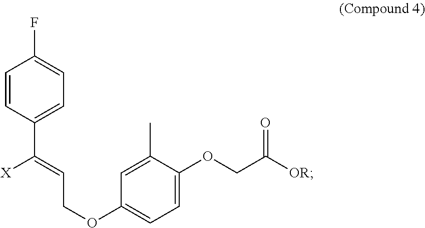

wherein R is C₁-C₆ alkyl; and
X is Br or I;
in the presence of a coupling catalyst, a suitable base, and in a suitable solvent, to provide Compound 5, or a salt thereof:

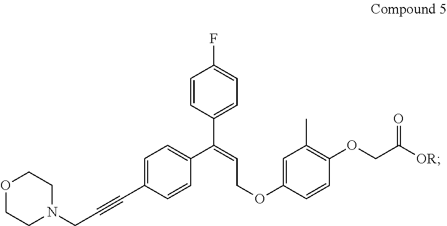

wherein R is C₁-C₆ alkyl;
(2) (i) reacting Compound 5 with sodium hydroxide, potassium hydroxide or lithium hydroxide in a suitable solvent to provide Compound 6:

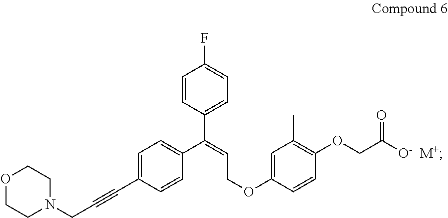

wherein M is sodium, potassium or lithium;
and
(ii) contacting Compound 6 with a suitable acid in a suitable solvent to provide Compound I:

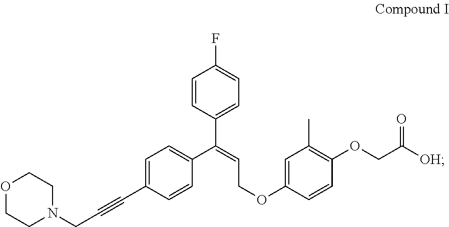 [SMILES: Cc1cc(OC/C=C(/c2ccc(F)cc2)c2ccc(C#CCN3CCOCC3)cc2)ccc1OCC(=O)O]

and
(3) reacting Compound I with a sodium hydroxide solution in the presence of a suitable solvent to provide Compound II.

In some embodiments, provided is a process for the preparation of Compound 5, or a salt thereof:

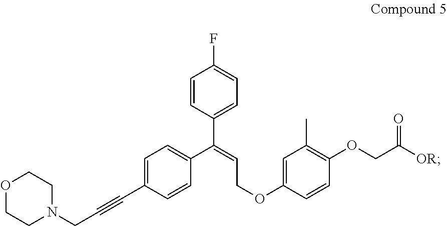

wherein R is C₁-C₆ alkyl;
comprising:
reacting Compound 3, or a salt thereof:

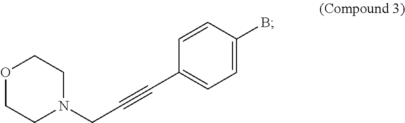

wherein B is a boronic acid, boronate ester, or trifluoroborate;
with Compound 4:

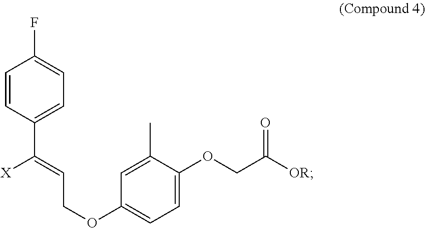

wherein R is C₁-C₆ alkyl; and
X is Br or I;
   in the presence of a suitable coupling catalyst, a suitable base, and in a suitable solvent, to provide Compound 5.

In some embodiments, provided is a process for the preparation of Compound 3, or salt thereof:

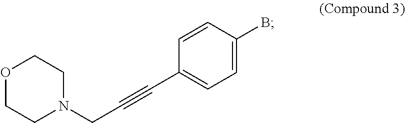

wherein B is a boronic acid, boronate ester, or trifluoroborate;
comprising reacting Compound 1:

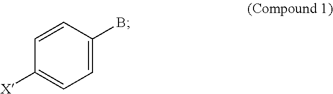

wherein X′ is Cl, Br or I;
with Compound 2, or salt thereof:

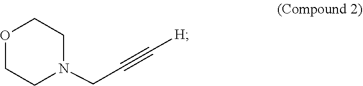

in the presence of a coupling catalyst, a suitable copper(I) cocatalyst, a suitable base, and in a suitable solvent.

In some embodiments, provided is the compound 4-(3-(4-(4,4,5,5-tetramethyl-1,3,2-dioxaborolan-2-yl)phenyl)prop-2-yn-1-yl)morpholine hydrochloride (Compound 3b):

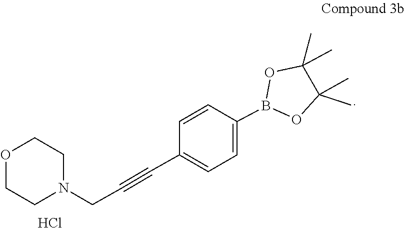 [SMILES: CC1(C)OB(c2ccc(C#CCN3CCOCC3)cc2)OC1(C)C ; Cl]

In some embodiments, provided is the compound having the following structure of Compound 4c:

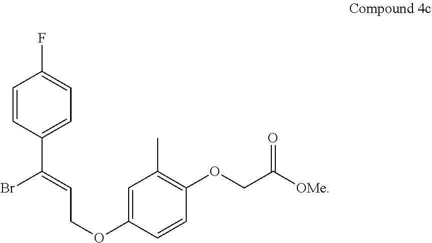 [SMILES: COC(=O)COc1ccc(OC/C=C(\Br)c2ccc(F)cc2)cc1C]

In some embodiments, provided is a process for the preparation of Compound 4c:

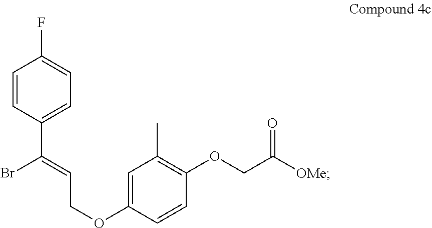 [SMILES: COC(=O)COc1ccc(OC/C=C(\Br)c2ccc(F)cc2)cc1C]

comprising:
reacting Compound 4-8:

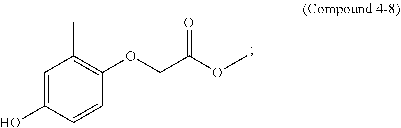

with Compound 4-4c:

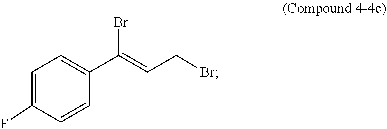 [SMILES: Fc1ccc(/C(Br)=C/CBr)cc1]

in the presence of a suitable base and in a suitable solvent to provide Compound 4c.

In some embodiments, provided is a process for the preparation of the Compound 4a:

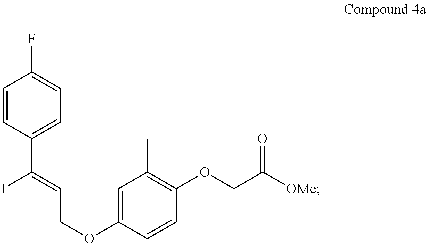 [SMILES: COC(=O)COc1ccc(OC/C=C(\I)c2ccc(F)cc2)cc1C]

comprising:
reacting Compound 4-8:

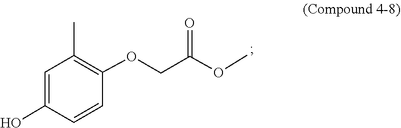

with Compound 4-4a:

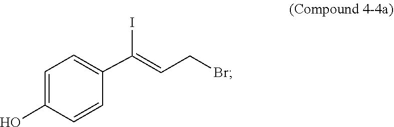 [SMILES: Oc1ccc(/C(I)=C/CBr)cc1]

in the presence of a suitable base and in a suitable solvent to provide Compound 4a.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

(E)-2-(4-((3-(4-Fluorophenyl)-3-(4-(3-morpholinoprop-1-yn-1-yl)phenyl)allyl)oxy)-2-methylphenoxy)acetic acid (Compound I) is a potent, selective and orally bioavailable PPARδ agonist. The PPARs are members of the nuclear receptor superfamily, which are ligand-modulated transcription factors that regulate gene expression of many cellular processes. The three PPARs, α, γ, and δ, are activated by lipids and are targets for current drug therapies for components of the metabolic syndrome. PPARα, a target for the fibrate class of triglyceride (TG)-lowering drugs, is primarily expressed in liver, where it upregulates genes involved in lipid oxidation in the fasted state. PPARγ is highly expressed in adipose tissue and regulates adipogenesis and insulin sensitivity. Pioglitazone is a drug from the thiazolidinedione class that increase insulin sensitivity through activating PPARγ. Compound I exhibits a significantly greater selectivity for PPARδ over PPARα and PPARγ (by 100-fold and 400-fold, respectively), and acts as a full agonist of PPARδ and only a partial agonist for both PPARα and PPARγ.

PPARδ controls genes involved in cellular metabolic processes such as glucose homeostasis, fatty acid synthesis and storage, and fatty acid mobilization and metabolism. PPARδ is expressed in several metabolically active tissues including liver, muscle, and fat. It is the most abundant PPAR isoform in skeletal muscle and has a higher expression in oxidative type I muscle fibers compared with glycolytic type II muscle fibers. A number of different physiological and pathological factors are reported to influence skeletal muscle PPARδ content. Both short term exercise and endurance training lead to increased PPARδ expression in human and rodent skeletal muscle. There is currently no marketed drug available targeting PPARδ.

Both genetic overexpression and pharmacological activation of PPARδ in mouse muscles results in increased number of fibers with high mitochondrial content and improves fatty acid oxidation. Overexpression of a constitutively active PPARδ (VP16-PPARδ) in skeletal muscles of transgenic mice pre-programs an increase in oxidative muscle fibers, enhancing running endurance in untrained adult mice (Wang, Y.-X., et al. (2004). Regulation of muscle fiber type and running endurance by PPARdelta. PLoS Biol. 2, e294). The PPARδ agonist, GW1516, in combination with exercise (for 4 weeks) synergistically induced fatigue-resistant oxidative muscle fibers and mitochondrial biogenesis in mice, and therefore enhanced physical performance (Narkar, V. A., et al. (2008). AMPK and PPARδ agonists are exercise mimetics. Cell 134, 405-415). When mice were treated with GW1516 for a longer time (8 weeks compared to 4 weeks) a clear shift in energy substrate usage from glucose to fatty acid oxidation to a level similar to exercise training was observed, indicative of increased fatty acid metabolism (Fan, W., et al. (2017). PPARδ Promotes Running Endurance by Preserving Glucose. Cell Metab. 25, 1186-1193.e4).

### Compound I

Compound I is a PPARδ agonist that is useful in the methods of treatment described herein. In human cell lines expressing all three peroxisome proliferator-activated receptor (PPAR) isotypes, Compound I is a potent (EC₅₀<100 nM) and selective human PPARδ agonist, with minor activity on PPARα (EC₅₀>10 μM) and PPARγ (EC₅₀>10 μM). Compound I is a full PPARδ agonist whereas it demonstrates only partial agonist activity on PPARα and PPARγ. Additionally, Compound I did not result in activation of human cells expressing the nuclear receptors RXR, FXR, LXR_{α} or LXR_{β}.

In vivo experiments demonstrated that Compound I treatment altered the expression patterns of several well-known PPARδ regulated genes in pathways involved in the beta-oxidation of long chain fatty acids (CPT1b) and mitochondrial biogenesis (PGC-1α) in mice muscle. In rat muscle, Compound I treatment increased the expression of a known PPAR regulated target gene, Angiopoietin-like 4 (ANGPTL4).

Compound I, or a pharmaceutically acceptable salt, or solvate, of hydrate thereof, was considered safe and well tolerated in clinical studies conducted to date. No serious adverse events (SAEs) were reported, and the incidence of adverse events (AEs) were similar between Compound I, or a pharmaceutically acceptable salt, or solvate, of hydrate thereof, treated and placebo groups.

Compound I refers to (E)-2-(4-((3-(4-fluorophenyl)-3-(4-(3-morpholinoprop-1-yn-1-yl)phenyl)allyl)oxy)-2-methylphenoxy)acetic acid, which has the chemical structure shown below.

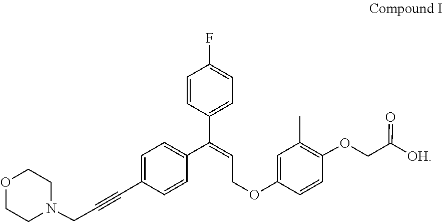 [SMILES: Cc1cc(OC/C=C(/c2ccc(F)cc2)c2ccc(C#CCN3CCOCC3)cc2)ccc1OCC(=O)O]

Compound II refers to sodium (E)-2-(4-((3-(4-fluorophenyl)-3-(4-(3-morpholinoprop-1-yn-1-yl)phenyl)allyl)oxy)-2-methylphenoxy)acetate, which has the chemical structure shown below.

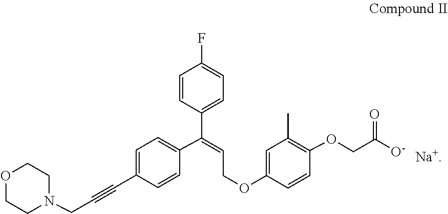 [SMILES: Cc1cc(OC/C=C(/c2ccc(F)cc2)c2ccc(C#CCN3CCOCC3)cc2)ccc1OCC(=O)[O-]]

In some embodiments, Compound II is amorphous.

In some embodiments, Compound II is crystalline.

### Synthesis

Compounds described herein are synthesized using standard synthetic techniques or using methods known in the art in combination with methods described herein. Unless otherwise indicated, conventional methods of mass spectroscopy, NMR, HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In the reactions described, it may be necessary to protect reactive functional groups, for example hydroxy or amino groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Synthesis of Compound I and Compound II

Disclosed herein are methods for the synthesis of Compound I and Compound II as outlined in Scheme A.

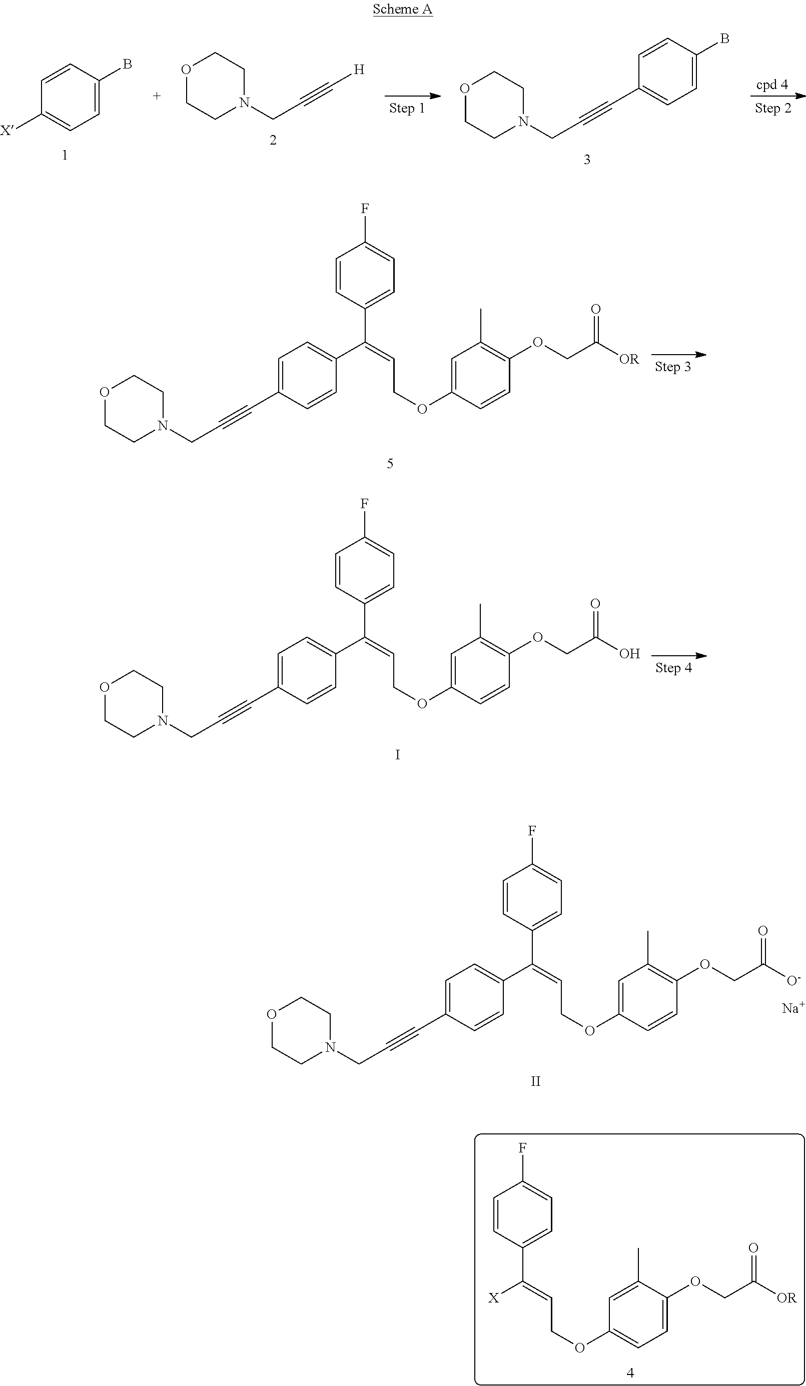

As disclosed herein, variables in Scheme A are defined as follows: B is a boronic acid, boronate ester, or trifluoroborate; X′ is Cl, Br or I; R is C₁-C₂₀ alkyl, C₁-C₂₀ alkenyl, C₃-C₁₀ cycloalkyl, or C₃-C₁₀ cycloalkenyl; and X is Br or I.

In some embodiments, Sonogashira cross-coupling of Compound 1 and Compound 2, or a salt thereof, in Step 1 yields Compound 3, or salt thereof. In some embodiments, subsequent Suzuki-Miyaura cross-coupling of the compound or salt of Compound 3, with the vinyl halide Compound 4 in Step 2 yields Compound 5, or a salt thereof. In some embodiments, after Step 2 and before Step 3, residual metal (e.g., palladium) is removed from Compound 5 by a metal scavenger. In some embodiments, saponification of the compounds or salt of Compound 5 in Step 3, followed by acid neutralization, yields the carboxylic acid Compound I. In some embodiments, Compound I is treated with a sodium solution (e.g., sodium hydroxide) to yield compound II. In some embodiments, compound II is crystallized.

### Step 1: Synthesis of Compound 3

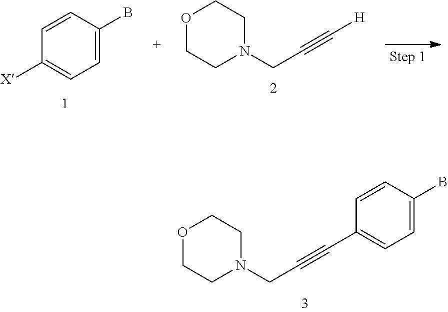

As disclosed herein, Compound 3, or salt thereof, is prepared from Compound 1 and Compound 2, or salt thereof. In some embodiments, Compound 3, or salt thereof, is produced by a Sonogashira cross-coupling of Compound 1 and Compound 2, or a salt thereof. In some embodiments, Compound 1 is reacted with Compound 2, or salt thereof, in the presence of a coupling catalyst, a suitable copper(I) cocatalyst, a suitable base, and in a suitable solvent to yield Compound 3, or salt thereof.

In some embodiments, the coupling catalyst in Step 1 is a palladium catalyst. In some embodiments, the palladium catalyst is a palladium(0) catalyst. In other embodiments, the palladium catalyst is a palladium(II) catalyst. In some embodiments, the palladium catalyst is precoordinated with a ligand. In some embodiments, Step 1 further comprises adding an exogenous ligand. In some embodiments, the ligand is a phosphine ligand. In some embodiments, the ligand is an aliphatic phosphine ligand, such as trimethyl phosphine, tricyclohexylphosphine, tri-tert-butyl-phosphine or the like. In some embodiments, the ligand is an aromatic phosphine, such as XPhos, SPhos, JohnPhos, Amphos, triphenylphosphine, methyldiphenylphosphine, or the like. In some embodiments, the ligand is a phosphite ligand, such as trimethylphosphite, triphenylphosphite, or the like. In some embodiments, the ligand is a bis-phosphine ligand, such as diphenylphosphinomethane (dppm), diphenyl phosphinoethane (dppe), 1,1′-bis(diphenylphosphino)ferrocene (dppf), or the like. In some embodiments, the ligand is triphenylphosphine. In some embodiments, the palladium catalyst is Pd(PPh₃)₂Cl₂. In some embodiments, the palladium catalyst is Pd(PPh₃)₃Cl. In some embodiments, the palladium catalyst is Pd(PPh₃)₄. In some embodiments, the amount of palladium used in Step 1 is from about 0.005 equiv to about 0.1 equiv. In some embodiments, the amount of palladium used in Step 1 is about 0.005, 0.01, 0.02, 0.03, 0.04, 0.05, 0.06, 0.07, 0.08, 0.09, or 0.1 equiv. In some embodiments, the amount of palladium used in Step 1 is about 0.01 equiv.

In some embodiments, the copper(I) cocatalyst in Step 1 is a copper(I) salt. In some embodiments, the copper(I) cocatalyst in Step 1 is CuCl, CuBr, or CuI. In some embodiments, the copper(I) cocatalyst is CuI. In some embodiments, the copper(I) cocatalyst is a copper(I)-N-heterocyclic carbene (Copper-NHC) complex. In some embodiments, the amount of copper(I) cocatalyst used in Step 1 is from about 0.001 equiv to about 0.1 equiv. In some embodiments, the amount of copper(I) cocatalyst used in Step 1 is about 0.001, about 0.002, about 0.003, about 0.004, about 0.005, about 0.01, about 0.02, about 0.03, about 0.04, about 0.05, about 0.06, about 0.07, about 0.08, about 0.09, or about 0.1 equiv. In some embodiments, the amount of copper(I) cocatalyst used in Step 1 is about 0.005 equiv.

In some embodiments, suitable bases in Sonogashira reactions include amine bases. In some embodiments, suitable amine bases for Sonogashira reactions are tertiary amine bases. Suitable amine bases for Sonogashira reactions include, but are not limited to, triethylamine, diisopropylethylamine, 1,2,2,6,6-pentamethylpiperidine, tributylamine, 1,8-diazabicycloundec-7-ene (DBU), or the like. In some embodiments, the base used in Step 1 is triethylamine. In some embodiments, the base used in Step 1 is 1,8-diazabicycloundec-7-ene (DBU). In some embodiments, about 1, 2, 3, 4, 5, or 6 equivalents of the base is used in Step 1. In some embodiments, about 1.5, about 2.5, about 3.5, about 4.5, about 5.5, or about 6.5 equivalents of the base is used in Step 1. In some embodiments, about 2.5 equivalents of the base is used in Step 1.

In some embodiments, the solvent system used in Step 1 is a single solvent. In some embodiments, the solvent system used in Step 1 is a cosolvent mixture. In some embodiments, the solvent system used in Step 1 is acetonitrile, dimethylformamide, diethyl ether, ethanol, tetrahydrofuran, 2-methyltetrahydrofuran, isopropyl alcohol, 1,4-dioxane, toluene, water, or a combination thereof. In some embodiments, the solvent system used in Step 1 is tetrahydrofuran.

In some embodiments, the temperature used in Step 1 is between about 40° and 100° C., preferably between about 50° C. and 70° C. In some embodiments, the temperature used in Step 1 is between 55° C. and 65° C. In some embodiments, the temperature used in Step 1 is between about 58° C. and about 63° C. In some embodiments, the temperature used in Step 1 is about 60° C.

In some embodiments, the B group in Compound 1 is a boronic acid or a boronic ester. In some embodiments, B is

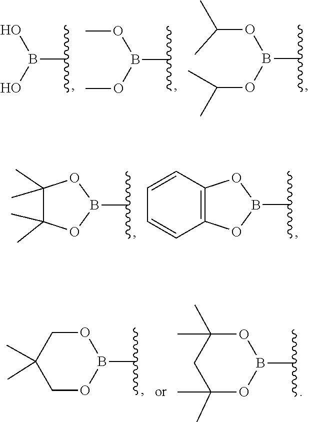

In some embodiments, B is a boronic acid. In some embodiments, B is

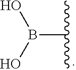 [SMILES: *B(O)O]

In some embodiments, B is a boronic ester. In some embodiments, B is

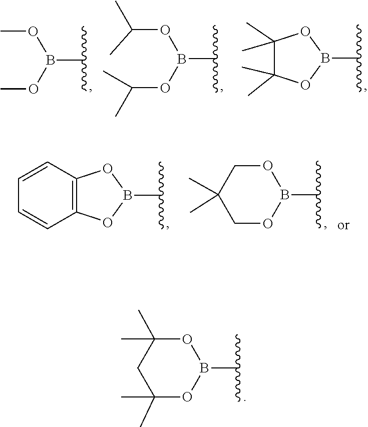

In some embodiments, B is

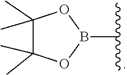 [SMILES: *B1OC(C)(C)C(C)(C)O1]

In some embodiments, B is a trifluoroborate. In some embodiments, B is

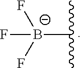 [SMILES: *[B-](F)(F)F]

In some embodiments, X′ is halogen in Compound 1. In some embodiments, X′ is Cl, Br, or I. In some embodiments, X′ is Br or I. In some embodiments, X′ is Br. In some embodiments, X′ is I.

In some embodiments, Compound 1 is Compound 1a:

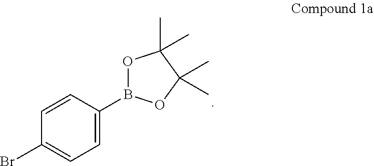 [SMILES: CC1(C)OB(c2ccc(Br)cc2)OC1(C)C]

In some embodiments, Compound 2, or a salt thereof, is used in the synthetic procedures described herein as a salt form or as a free base form. In some embodiments, the salt form of Compound 2 is an acid addition salt form. In some embodiments, a salt form of Compound 2 is used. In some embodiments, the hydrochloride salt of Compound 2 is used and is represented by Compound 2a:

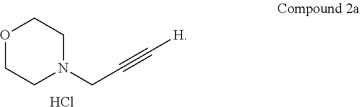

In some embodiments, Compound 3, or salt thereof, is isolated in free base form. In some embodiments, Compound 3, or salt thereof, is isolated as a salt form. In some embodiments, Compound 3, or salt thereof, is isolated as a hydrochloride salt. In some embodiments, Compound 3, or salt thereof, is Compound 3a, or salt thereof. In some embodiments, Compound 3, or salt thereof, is the hydrochloride salt Compound 3b.

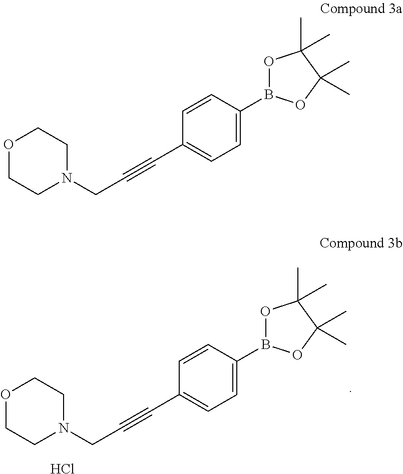 [SMILES: CC1(C)OB(c2ccc(C#CCN3CCOCC3)cc2)OC1(C)C ; CC1(C)OB(c2ccc(C#CCN3CCOCC3)cc2)OC1(C)C ; Cl]

### Step 2: Synthesis of the Compound 5

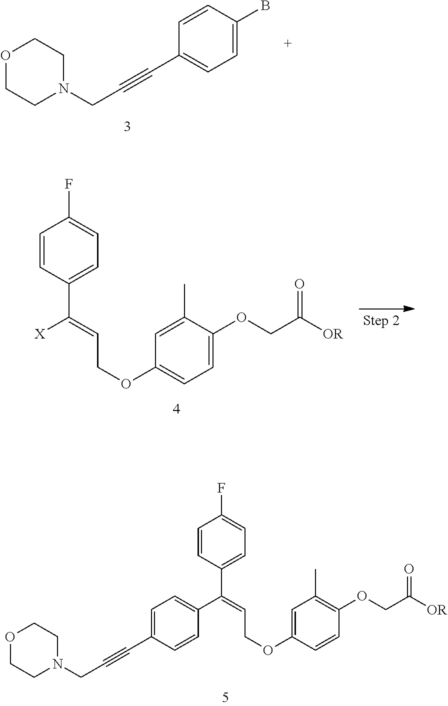

As disclosed herein, Compound 5, or salt thereof, is prepared from Compound 3, or salt thereof, and Compound 4. In some embodiments, Compound 5, or salt thereof, is produced by a Suzuki-Miyaura cross-coupling of Compound 3, or salt thereof, and Compound 4. In some embodiments, Compound 3, or salt thereof, is reacted with Compound 4, in the presence of a coupling catalyst, a suitable base, and in a suitable solvent to yield Compound 5, or salt thereof. In some embodiments, Compound 3, or salt thereof, in Step 2 is the hydrochloride salt hydrochloride salt Compound 3b.

In some embodiments, Compound 4 is Compound 4a, Compound 4b, Compound 4c, or Compound 4d:

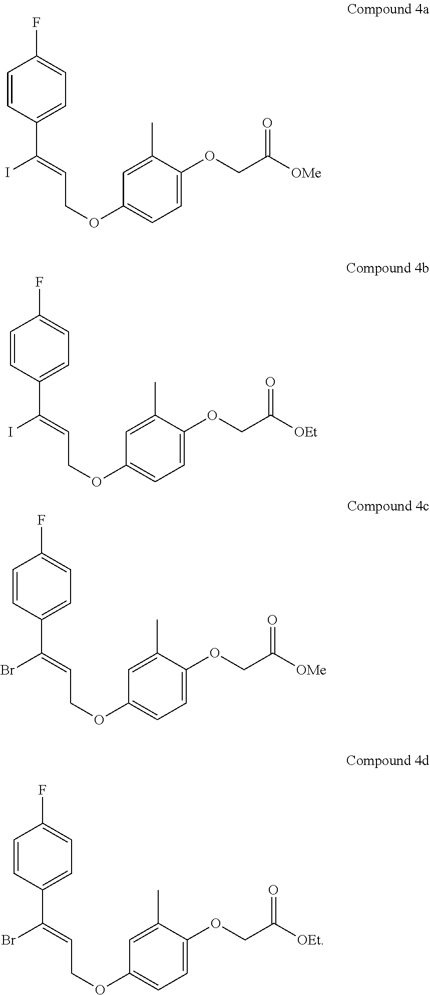 [SMILES: COC(=O)COc1ccc(OC/C=C(\I)c2ccc(F)cc2)cc1C ; CCOC(=O)COc1ccc(OC/C=C(\I)c2ccc(F)cc2)cc1C ; COC(=O)COc1ccc(OC/C=C(\Br)c2ccc(F)cc2)cc1C ; CCOC(=O)COc1ccc(OC/C=C(\Br)c2ccc(F)cc2)cc1C]

In some embodiments, Compound 4 is Compound 4a. In some embodiments, Compound 4 is Compound 4c.

In some embodiments, the coupling catalyst in Step 2 is a palladium catalyst. In some embodiments, the palladium catalyst is a palladium(0) catalyst. In other embodiments, the palladium catalyst is a palladium(II) catalyst. In some embodiments, the palladium catalyst is precoordinated with a ligand. In some embodiments, Step 2 further comprises adding an exogenous ligand. In some embodiments, the ligand is a phosphine ligand. In some embodiments, the ligand is an aliphatic phosphine ligand, such as trimethyl phosphine, tricyclohexylphosphine, tri-tert-butyl-phosphine or the like. In some embodiments, the ligand is an aromatic phosphine, such as XPhos, SPhos, JohnPhos, Amphos, triphenylphosphine, methyldiphenylphosphine, or the like. In some embodiments, the ligand is a phosphite ligand, such as trimethylphosphite, triphenylphosphite, or the like. In some embodiments, the ligand is a bis-phosphine ligand, such as diphenylphosphinomethane (dppm), diphenyl phosphinoethane (dppe), 1,1′-bis(diphenylphosphino)ferrocene (dppf), or the like. In some embodiments, the ligand is butyl di-1-adamantylphosphine. In some embodiments, the ligand is triphenylphosphine. In some embodiments, the palladium catalyst is Pd(PPh₃)₂Cl₂. In some embodiments, the palladium catalyst is Pd(PPh₃)₄. In some embodiments, the palladium catalyst is Pd₂(dba)₃. In some embodiments, the amount of palladium used in Step 2 is from about 0.005 equiv to about 0.1 equiv. In some embodiments, the amount of palladium used in Step 2 is about 0.005, 0.01, 0.02, 0.03, 0.04, 0.05, 0.06, 0.07, 0.08, 0.09, or 0.1 equiv. In some embodiments, the amount of palladium used in Step 2 is about 0.01 equiv. In some embodiments, the amount of palladium used in Step 2 is about 0.02 equiv. In some embodiments, the amount of palladium used in Step 2 is about 0.03 equiv.

In some embodiments, suitable bases in Suzuki reactions include amine bases and inorganic bases. Suitable amine bases for Suzuki reactions include, but are not limited to, triethylamine, diisopropylethylamine, 1,2,2,6,6-pentamethylpiperidine, tributylamine, 1,8-diazabicycloundec-7-ene (DBU), or the like. Suitable inorganic bases for Suzuki reactions include, but are not limited to, sodium bicarbonate, NaOAc, KOAc, Ba(OH)₂, Li₂CO₃, Na₂CO₃, K₂CO₃, Cs₂CO₃, Na₃PO₄, K₃PO₄, CsF, or the like. In some embodiments, the base used in Step 2 is CsF. In some embodiments, the base used in Step 2 is triethylamine. In some embodiments, the base used in Step 2 is Na₂CO₃. In some embodiments, the base used in Step 2 is K₂CO₃. In some embodiments, about 1, 2, 3, 4, 5, or 6 equivalents of the base is used in Step 2. In some embodiments, 1.1 equivalents of base is used in Step 2.

In some embodiments, the suitable solvent used in Step 2 is a single solvent. In some embodiments, the suitable solvent used in Step 2 is a cosolvent mixture. In some embodiments, the suitable solvent used in Step 2 is acetonitrile, dimethylformamide, dimethoxyethane, 2-methyltetrahydrofuran, methyl tert-butyl ether, cyclopentyl methyl ether, tetrahydrofuran, diisopropyl ether, 1,4-dioxane, toluene, water, or a combination thereof. In some embodiments, the suitable solvent used in Step 2 is a mixture of toluene and water. In some embodiments, the suitable solvent used in Step 2 is methyl tert-butyl ether (MTBE).

In some embodiments, the temperature used in Step 2 is between about 40° and 120° C., preferably between about 50° C. and 100° C. In some embodiments, the temperature used in Step 2 is between about 57° C. and about 62° C. In some embodiments, the temperature used in Step 2 is about 60° C. In some embodiments, the temperature used in Step 2 is about 80° C. In some embodiments, the temperature used in Step 2 is about 90° C. In some embodiments, the temperature used in Step 2 is between 77° C. and 82° C.

In some embodiments the B group of Compound 3, or salt thereof, is a boronic acid or a boronic ester. In some embodiments, B is

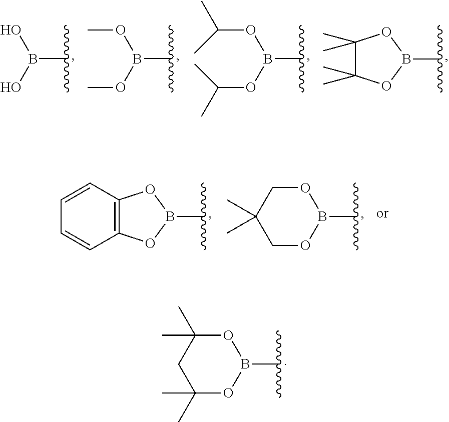

In some embodiments, B is a boronic acid. In some embodiments, B is

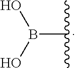 [SMILES: *B(O)O]

In some embodiments, B is a boronic ester. In some embodiments, B is

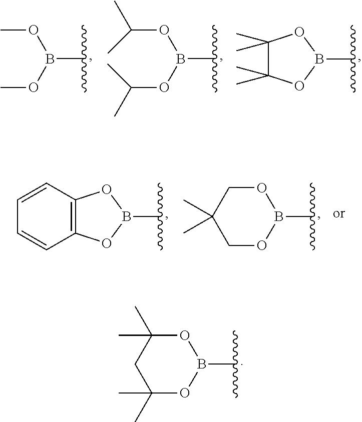

In some embodiments, B is

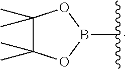 [SMILES: *B1OC(C)(C)C(C)(C)O1]

In some embodiments, B is a trifluoroborate. In some embodiments, B is

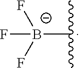 [SMILES: *[B-](F)(F)F]

In some embodiments, the X group of Compound 4 is a halogen. In some embodiments, X is Cl, Br, or I. In some embodiments, X is Br or I. In some embodiments, X is Br. In some embodiments, X is I.

In some embodiments, the R group of Compound 4 is C₁-C₂₀ alkyl, C₁-C₂₀ alkenyl, C₃-C₁₀ cycloalkyl, or C₃-C₁₀ cycloalkenyl. In some embodiments, R is C₁-C₁₀ alkyl or C₁-C₁₀ alkenyl. In some embodiments, R is methyl, ethyl, propyl, isopropyl, butyl, isobutyl, tert-butyl, isoamyl, pentyl, hexyl, heptyl, octyl, nonyl, terpenyl, bornyl, allyl, linalyl or geranyl. In some embodiments, R is C₁-C₁₀ alkyl. In some embodiments, R is C₁-C₆ alkyl. In some embodiments, R is C₁-C₄ alkyl. In some embodiments, R is methyl, ethyl, propyl, isopropyl, butyl, isobutyl, tert-butyl, isoamyl, pentyl, or hexyl. In some embodiments, R is methyl or ethyl. In some embodiments, R is methyl. In some embodiments, R is ethyl.

In some embodiments, Compound 5, or salt thereof, is used in the synthetic procedures described herein as a free base form. In some embodiments, Compound 5, or salt thereof, is used in the synthetic procedures described herein as a salt form. In some embodiments, a hydrochloride salt of Compound 5 is used.

In some embodiments, the R group of Compound 5, or salt thereof, is C₁-C₂₀ alkyl, C₁-C₂₀ alkenyl, C₃-C₁₀ cycloalkyl, or C₃-C₁₀ cycloalkenyl. In some embodiments, R is C₁-C₁₀ alkyl or C₁-C₁₀ alkenyl. In some embodiments, R is methyl, ethyl, propyl, isopropyl, butyl, isobutyl, tert-butyl, isoamyl, pentyl, hexyl, heptyl, octyl, nonyl, terpenyl, bornyl, allyl, linalyl or geranyl. In some embodiments, R is C₁-C₁₀ alkyl. In some embodiments, R is C₁-C₆ alkyl. In some embodiments, R is C₁-C₄alkyl. In some embodiments, R is methyl, ethyl, propyl, isopropyl, butyl, isobutyl, tert-butyl, isoamyl, pentyl, or hexyl. In some embodiments, R is methyl or ethyl. In some embodiments, R is methyl. In some embodiments, R is ethyl.

In some embodiments, Compound 5, or salt thereof, is Compound 5a, or salt thereof, Compound 5b, or salt thereof, the hydrochloride salt Compound 5c, or the hydrochloride salt Compound 5d:

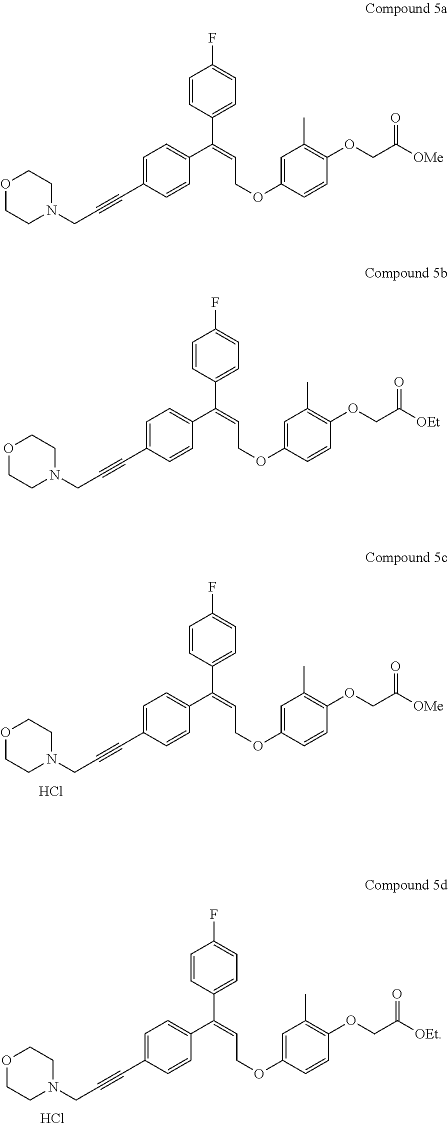 [SMILES: COC(=O)COc1ccc(OC/C=C(/c2ccc(F)cc2)c2ccc(C#CCN3CCOCC3)cc2)cc1C ; CCOC(=O)COc1ccc(OC/C=C(/c2ccc(F)cc2)c2ccc(C#CCN3CCOCC3)cc2)cc1C ; COC(=O)COc1ccc(OC/C=C(/c2ccc(F)cc2)c2ccc(C#CCN3CCOCC3)cc2)cc1C ; Cl ; CCOC(=O)COc1ccc(OC/C=C(/c2ccc(F)cc2)c2ccc(C#CCN3CCOCC3)cc2)cc1C ; Cl]

Due to the fact that the synthetic methods described above utilize a transition metal catalyst, purification steps are performed to reduce the amount of palladium in the product. Purification steps to reduce the amount of palladium in a product are conducted so that active pharmaceutical ingredients meet palladium specification guidelines. (“Guideline on the Specification Limits for Residues of Metal Catalysts” European Medicines Agency Pre-authorisation Evaluation of Medicines for Human Use, London, January 2007, Doc. Ref. CPMP/SWP/QWP/4446/00 corr.). In some embodiments, purification steps to reduce the amount of palladium in a product includes, but is not limited to, treatment with solid trimercaptotriazine (TMT), polystyrene-bound TMT, mercapto-porous polystyrene-bound TMT, polystyrene-bound ethylenediamine, activated carbon, glass bead sponges, Smopex™, silica bound scavengers, thiol-derivatized silica gel, N-acetylcysteine, n-Bu₃P, crystallization, extraction, L-cysteine, n-Bu₃P/lactic acid (Garrett et al., Adv. Synth. Catal. 2004, 346, 889-900). In some embodiments, activated carbon includes but is not limited to DARCO® KB-G, DARCO® KB-WJ. In one aspect silica bound scavengers include but are not limited to

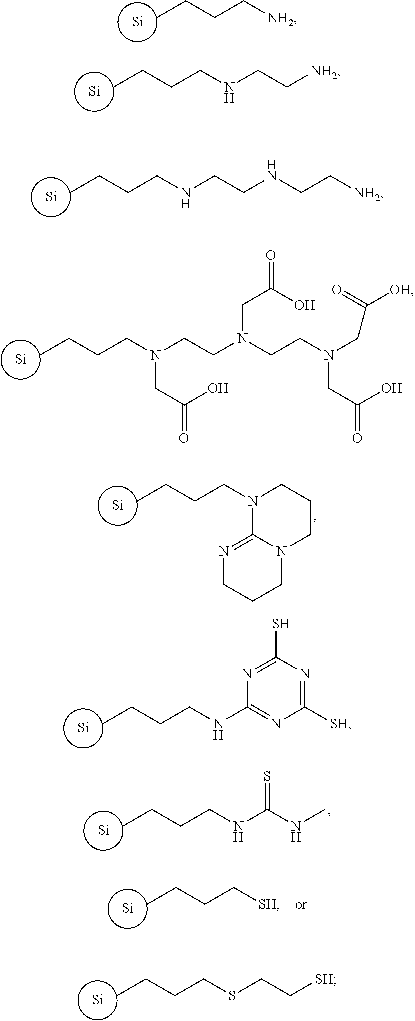

where

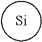

denotes silica gel. In some embodiments, the purification steps to reduce the amount of palladium include the use of activated carbon, derivatized silica gel (e.g., thiol derivatized silica gel), or combinations thereof.

In some embodiments, Compound 5, or salt thereof, is further treated with a metal scavenger to remove residual palladium. In some embodiments, the metal scavenger comprises SiO2, charcoal, aqueous solution of L-cysteine, a Silicycle metal scavenger, Si-thiol, SiliaBond DMT, SiliaBond Cysteine, or 3-mercaptopropyl ethyl sulfide silica. In some embodiments, the scavenger loading (w/w) is about 1:3, about 1:2, or about 1:1. In some embodiments, the metal scavenger is 3-mercaptopropyl ethyl sulfide silica. In some embodiments, the metal scavenger is L-cysteine.

In some of these embodiments, palladium levels are reduced to about 100 ppm or less. In some of these embodiments, palladium levels are reduced to about 10 ppm. In some of these embodiments, palladium levels are reduced sufficiently to be undetectable.

In some embodiments, the presence of residual heavy metal (e.g. palladium) impurities is determined by utilizing methods known in the art. In some embodiments, the presence of residual heavy metal (e.g. palladium) impurities is determined by the use of inductively coupled plasma mass spectrometry (ICP-MS). In some embodiments, the presence of residual heavy metal (e.g. palladium) impurities is determined by the use of techniques described in U.S. Pharmacopeia General Chapter <231> Heavy Metals.

### Step 3: Synthesis of Compound I

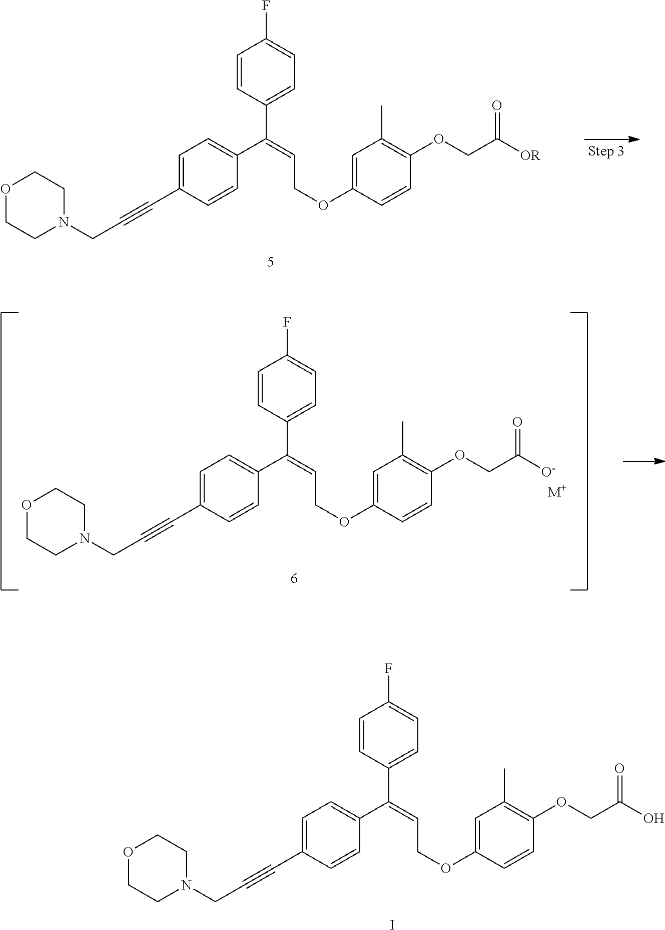

As disclosed herein, Compound I, or salt thereof, is prepared from Compound 5, or salt thereof. In some embodiments, saponification of the compounds or acid addition salt form of Compound 5 in Step 3, followed by acid neutralization, yields the carboxylic acid Compound I, or salt thereof. In some embodiments, Compound 5, or salt thereof, is reacted with sodium hydroxide, potassium hydroxide or lithium hydroxide in a suitable solvent to yield Compound 6. In some embodiments, treatment of Compound 6 with a suitable acid in a suitable solvent provides Compound I, or salt thereof. In some embodiments, Compound 6 is not isolated before treatment with the suitable acid in the suitable solvent.

In some embodiments, Compound 5, or salt thereof, is reacted with sodium hydroxide to provide Compound 6 wherein M⁺ is Na⁺ (i.e. Compound II). In other embodiments, Compound 5, or salt thereof, is reacted with potassium hydroxide to provide Compound 6 wherein M⁺ is K⁺. In other embodiments, Compound 5, or salt thereof, is reacted with lithium hydroxide to provide Compound 6 wherein M⁺ is Li⁺. In some embodiments, about 1, about 1.5, about 2, about 2.5, about 3, about 4, or about 5 equivalents of sodium hydroxide, potassium hydroxide or lithium hydroxide is used in Step 3. In some embodiments, about 2.5 equivalents of sodium hydroxide is used in Step 3.

In some embodiments, the suitable solvent used in Step 3 is a single solvent. In some embodiments, the suitable solvent used in Step 3 is a cosolvent mixture. In some embodiments, the suitable solvent used in Step 3 is water, methanol, ethanol, tetrahydrofuran, ethyl acetate, or a combination thereof. In some embodiments, the suitable solvent used in Step 3 is a mixture of ethanol and water.

In some embodiments, the temperature used in Step 3 is between about 0° C. and 50° C., preferably between about 15° C. and 30° C. In some embodiments, the temperature used in Step 3 is about 25° C. In some embodiments, the temperature used in Step 3 is between 15° C. and 25° C.

In some embodiments, the suitable acid for neutralization in Step 3 is acetic acid, citric acid, oxalic acid, lactic acid, hydrochloric acid, nitric acid, or sulfuric acid. In some embodiments, the suitable acid is acetic acid.

In some embodiments, the suitable solvent used in the neutralization step of Step 3 is a single solvent. In some embodiments, the suitable solvent is a cosolvent mixture. In some embodiments, the suitable solvent is water, methanol, ethanol, tetrahydrofuran, ethyl acetate, or a combination thereof. In some embodiments, the suitable solvent is water. In some embodiments, the suitable solvent is ethanol.

### Step 4: Synthesis of Compound II

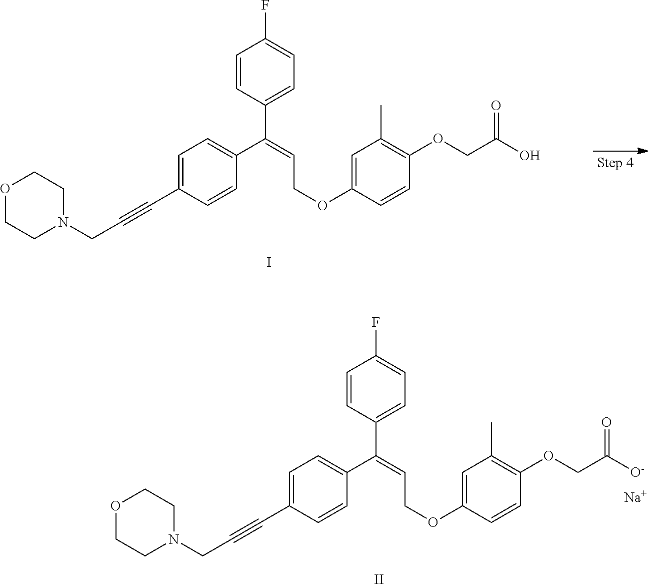

As disclosed herein, Compound II is prepared from Compound I, or salt thereof. In some embodiments, Compound I, or salt thereof, is treated with a sodium solution to yield compound II. In some embodiments, Compound I, or salt thereof, is treated with a sodium hydroxide solution in the presence of a suitable solvent to provide II.

In some embodiments, the suitable solvent used in Step 4 is a single solvent. In some embodiments, the suitable solvent is a cosolvent mixture. In some embodiments, the suitable solvent is water, methanol, ethanol, tetrahydrofuran, ethyl acetate, acetone, acetonitrile, or a combination thereof. In some embodiments, the suitable solvent is a mixture of water and ethyl acetate. In some embodiments, the suitable solvent is a mixture of water, ethanol, and ethyl acetate.

In some embodiments, the temperature used in Step 4 is between about 20° and 50° C. In some embodiments, the temperature used in Step 4 is about 40° C. In some embodiments, the temperature used in Step 4 is about 50° C.

### Synthesis of Intermediate Compound 4a

Also disclosed herein are methods for the synthesis of Compound 4a and Compound 4c, as outlined in Scheme B.

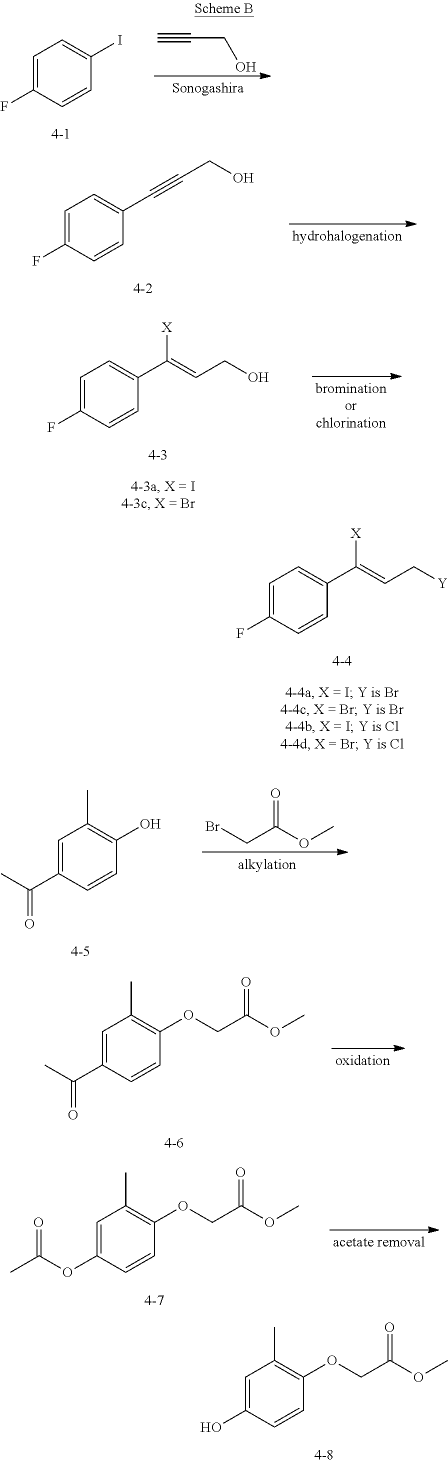

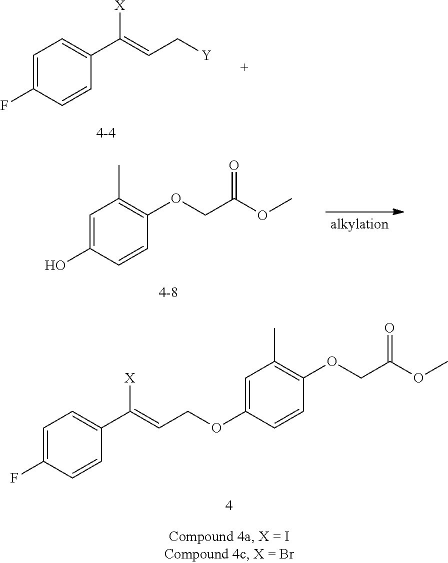

In some embodiments, Sonogashira cross-coupling of Compound 4-1 and propargyl alcohol yields Compound 4-2. In some embodiments, subsequent hydrohalogenation (e.g., hydroiodation, hydrobromination) of alkyne 4-2 yields Compound 4-3. In some embodiments, the allyl alcohol 4-3 is subsequently brominated or chlorinated to yield Compound 4-4.

The Sonogashira cross-coupling reaction between Compound 4-1 and propargyl alcohol is performed in the presence of a coupling catalyst, a suitable copper(I) cocatalyst, a suitable base, and in a suitable solvent to yield Compound 4-2 (vide supra for Step 1 in Scheme A). In some embodiments, the suitable coupling catalyst is Pd(PPh₃)₃Cl. In some embodiments, the suitable copper(I) cocatalyst is CuI. In some embodiments, the suitable base is diisopropylethylamine. In some embodiments, the suitable solvent is 2-methyltetrahydrofuran.

Hydrohalogenation of alkyne Compound 4-2 yields vinyl halide Compound 4-3 (e.g., vinyl iodide Compound 4-3a or vinyl bromide Compound 4-3c). In some embodiments, hydroiodation of alkyne Compound 4-2 yields vinyl iodide Compound 4-3a. In some embodiments, hydrobromination of alkyne Compound 4-2 yields vinyl bromide Compound 4-3c. In some embodiments, the reaction proceeds through a first step of hydrometalation before addition of an iodonium (I⁺) source in a suitable solvent. In some embodiments, the reaction proceeds through a first step of hydrometalation before addition of a bromonium (Br⁺) source in a suitable solvent. In some embodiments, hydrometalation is performed by a metal hydride. In some embodiments, the metal hydride is an aluminum hydride. In some embodiments, the metal hydride is lithium aluminum hydride (LAH), diisobutylaluminum hydride (DIBAL), or the like. In some embodiments, the iodonium source is iodine (I₂), N-iodosuccinimide (NIS), or the like. In some embodiments, the bromonium source is bromine (Br₂), N-bromosuccinimide (NBS), or the like. In some embodiments, the suitable solvent used in the hydroiodation or hydrobromination step is dimethoxyethane, 2-methyltetrahydrofuran, methyl tert-butyl ether, cyclopentyl methyl ether, tetrahydrofuran, diisopropyl ether, 1,4-dioxane, or a combination thereof. In some embodiments, the suitable solvent used in the hydroiodation or hydrobromination step is 2-methyltetrahydrofuran. In some embodiments, the suitable solvent used in the hydroiodation or hydrobromination step is tetrahydrofuran. In some embodiments, the suitable solvent used in the hydroiodination or hydrobromination step is a mixture of 2-methyltetrahydrofuran and tetrahydrofuran.

Bromination of allylic alcohol Compound 4-3 yields Compound 4-4, wherein Y is Br. In some embodiments, Compound 4-4 is Compound 4-4a. In some embodiments, Compound 4-4 is Compound 4-4c. In some embodiments, Compound 4-3 (i.e., Compound 4-3a or Compound 4-3c) is reacted with a suitable brominating agent in a suitable solvent to yield Compound 4-4 (e.g., Compound 4-4a or Compound 4-4c). In some embodiments, the suitable brominating agent is PBr₃, PPh₃ and N-bromosuccinimide (NBS), PPh₃ and CBr₄, PPh₃ and Br₂, or the like. In some embodiments, the suitable solvent used in the bromination step is dimethoxyethane, 2-methyltetrahydrofuran, methyl tert-butyl ether, cyclopentyl methyl ether, tetrahydrofuran, diisopropyl ether, 1,4-dioxane, dichloromethane, toluene, or a combination thereof. In some embodiments, the suitable solvent used in the bromination step is dichloromethane.

Chlorination of allylic alcohol Compound 4-3 yields allyl bromide Compound 4-4, wherein Y is Cl. In some embodiments, Compound 4-4 is Compound 4-4b. In some embodiments, Compound 4-4 is Compound 4-4d. In some embodiments, Compound 4-3 (e.g., Compound 4-3a or Compound 4-3c) is reacted under suitable chlorination conditions in a suitable solvent to yield Compound 4-4 (i.e., Compound 4-4a or Compound 4-4c). In some embodiments, the suitable chlorinating agent is thionyl chloride, oxalyl chloride, methanesulfonyl chloride, arylsulfonyl chloride (e.g. benzenesulfonyl chloride, toluenesulfonyl chloride), or the like. In some embodiments, chlorination conditions comprise the use of a suitable base. In some embodiments, the suitable base is an amine base. Suitable amine bases include, but are not limited to, triethylamine, diisopropylethylamine, N-methylmorpholine, pyridine, 4-(dimethylamino)pyridine, dabco, 1,5-diazabicyclo[4.3.0]non-5-ene, and 1,4-diazabicyclo[2.2.2]octane. In some embodiments, the suitable solvent is dimethoxyethane, 2-methyltetrahydrofuran, methyl tert-butyl ether, cyclopentyl methyl ether, tetrahydrofuran, diisopropyl ether, 1,4-dioxane, dichloromethane, toluene, or a combination thereof.

In some embodiments, alkylation of Compound 4-5 with methyl 2-bromoacetate yields Compound 4-6. In some embodiments, Baeyer-Villiger oxidation of the ketone 4-6 yields Compound 4-7, and subsequent removal of the acetate group yields Compound 4-8. In some embodiments, Compound 4-8 is alkylated with Compound 4-4 to yield Compound 4a or Compound 4c.

Alkylation of Compound 4-5 with methyl 2-bromoacetate with a suitable base in a suitable solvent yields Compound 4-6. In some embodiments, the suitable base is sodium bicarbonate, NaOAc, KOAc, Ba(OH)₂, Li₂CO₃, Na₂CO₃, K₂CO₃, Cs₂CO₃, Na₃PO₄, K₃PO₄, CsF, or the like. In some embodiments, the suitable base is Cs₂CO₃. In some embodiments, the suitable solvent used in the alkylation step is acetonitrile, dimethylformamide, dimethoxyethane, 2-methyltetrahydrofuran, methyl tert-butyl ether, cyclopentyl methyl ether, tetrahydrofuran, diisopropyl ether, 1,4-dioxane, toluene, or a combination thereof. In some embodiments, the suitable solvent used in the alkylation step is acetonitrile.

Baeyer-Villiger oxidation of the ketone Compound 4-6 yields Compound 4-7. In some embodiments, treatment of ketone 4-6 with a suitable oxidant in a suitable solvent yields Compound 4-7. In some embodiments, treatment of ketone Compound 4-6 with a suitable peroxyacid or peroxide in a suitable solvent yields Compound 4-7. In some embodiments, the suitable peroxyacid or peroxide is meta-chloroperbenzoic acid (m-CPBA), peracetic acid, trifluoroperacetic acid, oxone, hydrogen peroxide, or the like. In some embodiments, the suitable peroxyacid or peroxide is m-CPBA. In some embodiments, the suitable solvent used in the Baeyer-Villiger oxidation step is trifluoroacetic acid, dichloromethane, acetonitrile, dimethylformamide, dimethoxyethane, ethyl acetate, methanol, water, toluene, or a combination thereof. In some embodiments, the suitable solvent used in the Baeyer-Villiger oxidation step is dichloromethane.

The removal of the acetate group of Compound 4-7 is performed in the presence of a suitable base and in a suitable solvent to yield Compound 4-8. In some embodiments, the suitable base is NaOH, LiOH, NaOAc, KOAc, Li₂CO₃, Na₂CO₃, K₂CO₃, Cs₂CO₃, or the like. In some embodiments, the suitable base used in the deprotection step is NaOH. In some embodiments, the suitable base used in the deprotection step is Na₂CO₃. In some embodiments, the suitable base used in the deprotection step is K₂CO₃. In some embodiments, the suitable solvent used in the deprotection step is acetonitrile, methanol, ethanol, tetrahydrofuran, isopropyl alcohol, isopropyl acetate, 1,4-dioxane, toluene, water, or a combination thereof. In some embodiments, the suitable solvent used in the deprotection step is acetonitrile. In some embodiments, the suitable solvent used in the deprotection step is methanol.

Alkylation of Compound 4-8 with Compound 4-4 with a suitable base and in a suitable solvent yields Compound 4a. Alkylation of Compound 4-8 with Compound 4-4c with a suitable base and in a suitable solvent yields Compound 4c. In some embodiments, the suitable base is sodium bicarbonate, NaOAc, KOAc, Ba(OH)₂, Li₂CO₃, Na₂CO₃, K₂CO₃, Cs₂CO₃, Na₃PO₄, K₃PO₄, CsF, or the like. In some embodiments, the suitable base is Cs₂CO₃. In some embodiments, the suitable base is K₂CO₃. In some embodiments, the suitable base is Na₂CO₃. In some embodiments, the suitable solvent used in the alkylation step is acetonitrile, dimethylformamide, dimethoxyethane, 2-methyltetrahydrofuran, methyl tert-butyl ether, cyclopentyl methyl ether, tetrahydrofuran, diisopropyl ether, 1,4-dioxane, toluene, or a combination thereof. In some embodiments, the suitable solvent used in the alkylation step is acetonitrile. In some embodiments, the solvent used in the alkylation step is methyl tert-butyl ether. In some embodiments, the solvent used in the alkylation step is a combination of methyl tert-butyl ether and water.

In some embodiments, the alkylation of Compound 4-8 with Compound 4-4 is performed at a temperature between about 40° C. and about 100° C. In some embodiments, the alkylation step is performed at a temperature between about 50° C. and about 80° C. In some embodiments, the alkylation step is performed at a temperature between about 57° C. and about 62° C. In some embodiments, the alkylation step is performed at about 50° C., about 60° C., about 70° C., or about 80° C. In some embodiments, the alkylation step is performed at about 60° C.

### Alternative Synthesis of Compound II

Also disclosed herein are methods for an alternative synthesis of Compound II, as outlined in Scheme C.

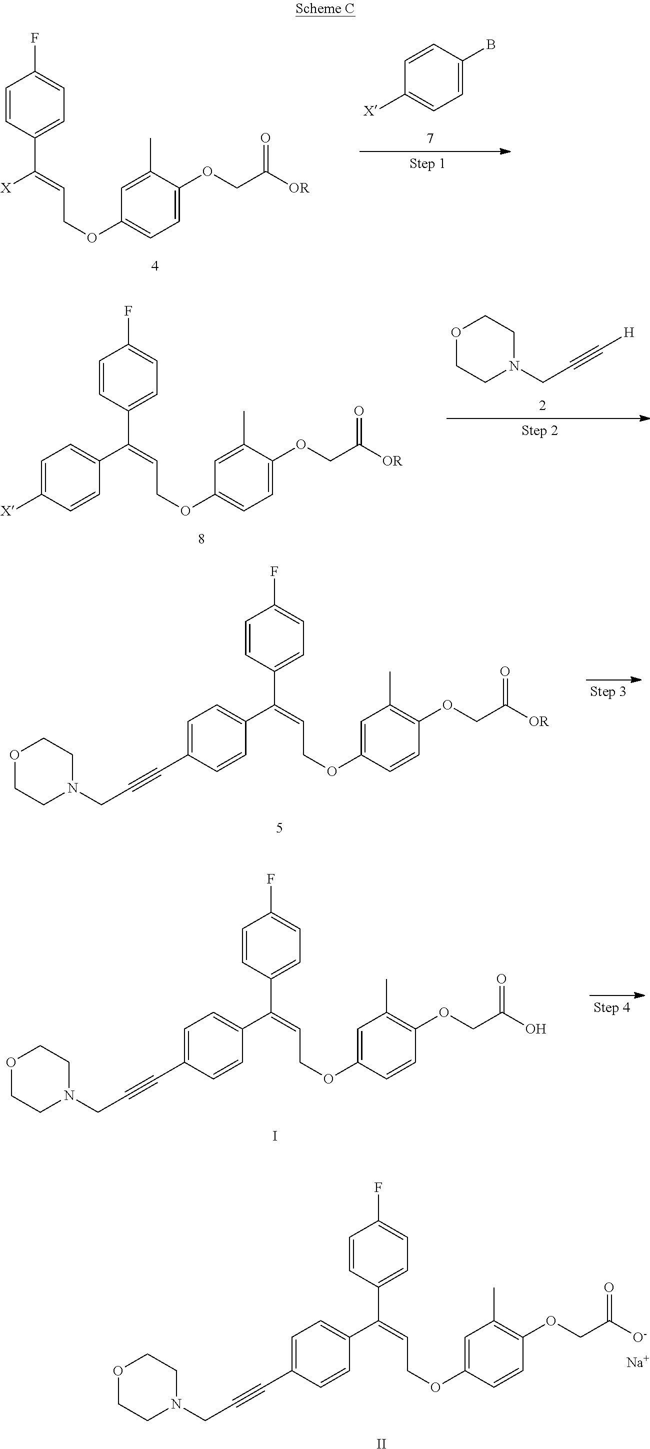

As disclosed herein, variables in Scheme C are defined as follows: R is C₁-C₂₀ alkyl, C₁-C₂₀ alkenyl, C₃-C₁₀ cycloalkyl, or C₃-C₁₀ cycloalkenyl; and X is Br or I; B is a boronic acid, boronate ester, or trifluoroborate; and X′ is Cl, Br or I.

In some embodiments, Suzuki-Miyaura cross-coupling of the vinyl halide Compound 4 with Compound 7 in Step 1 yields Compound 8. In some embodiments, subsequent Sonogashira cross-coupling of Compound 8 and Compound 2, or a salt thereof, in Step 2 yields Compound 5, or salt thereof. In some embodiments, after Step 2 and before Step 3, residual metal (e.g., palladium) is removed from Compound 5, or a salt thereof, by a metal scavenger. In some embodiments, the final two steps of the synthesis follow the same steps as described above for Scheme A. In some embodiments, saponification of the compounds or acid addition salt of Compound 5 in Step 3, followed by acid neutralization, yields Compound I. In some embodiments, Compound I is treated with a basic solution (e.g., sodium hydroxide) to yield compound II. In some embodiments, compound II is crystallized.

### Step 1: Synthesis of the Compound 8

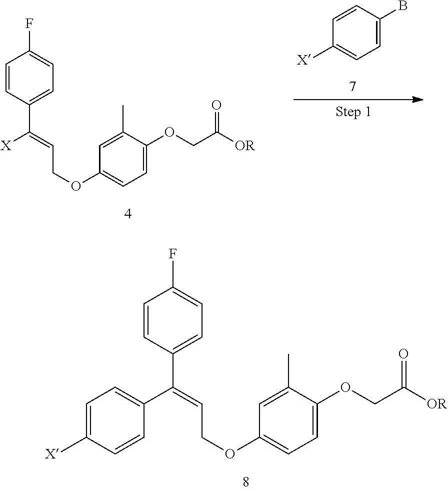

As disclosed herein, Compound 8 is prepared from Compound 4 and Compound 7. In some embodiments, Compound 8 is produced by a Suzuki-Miyaura cross-coupling of Compound 4 and Compound 7. In some embodiments, Compound 4 is reacted with Compound 7 in the presence of a coupling catalyst, a suitable base, and in a suitable solvent to yield Compound 8.

In some embodiments, the coupling catalyst in Step 1 is a palladium catalyst. In some embodiments, the palladium catalyst is a palladium(0) catalyst. In other embodiments, the palladium catalyst is a palladium(II) catalyst. In some embodiments, the palladium catalyst is precoordinated with a ligand. In some embodiments, Step 1 further comprises adding an exogenous ligand. In some embodiments, the ligand is a phosphine ligand. In some embodiments, the ligand is an aliphatic phosphine ligand, such as trimethyl phosphine, tricyclohexylphosphine, tri-tert-butyl-phosphine or the like. In some embodiments, the ligand is an aromatic phosphine, such as XPhos, SPhos, JohnPhos, Amphos, triphenylphosphine, methyldiphenylphosphine, or the like. In some embodiments, the ligand is a phosphite ligand, such as trimethylphosphite, triphenylphosphite, or the like. In some embodiments, the ligand is a bis-phosphine ligand, such as diphenylphosphinomethane (dppm), diphenyl phosphinoethane (dppe), 1,1′-bis(diphenylphosphino)ferrocene (dppf), or the like. In some embodiments, the ligand is triphenylphosphine. In some embodiments, the palladium catalyst is Pd(PPh₃)₂Cl₂. In some embodiments, the palladium catalyst is Pd(PPh₃)₄. In some embodiments, the amount of palladium used in Step 1 is from about 0.005 equiv to about 0.1 equiv. In some embodiments, the amount of palladium used in Step 1 is about 0.005, 0.01, 0.02, 0.03, 0.04, 0.05, 0.06, 0.07, 0.08, 0.09, or 0.1 equiv. In some embodiments, the amount of palladium used in Step 1 is about 0.01 equiv. In some embodiments, the amount of palladium used in Step 1 is about 0.02 equiv. In some embodiments, the amount of palladium used in Step 1 is about 0.03 equiv.

In some embodiments, suitable bases in Suzuki reactions include amine bases and inorganic bases. Suitable amine bases for Suzuki reactions include, but are not limited to, triethylamine, diisopropylethylamine, 1,2,2,6,6-pentamethylpiperidine, tributylamine, 1,8-diazabicycloundec-7-ene (DBU), or the like. Suitable inorganic bases for Suzuki reactions include, but are not limited to, sodium bicarbonate, NaOAc, KOAc, Ba(OH)₂, Li₂CO₃, Na₂CO₃, K₂CO₃, Cs₂CO₃, Na₃PO₄, K₃PO₄, CsF, or the like. In some embodiments, the base used in Step 1 is CsF. In some embodiments, the base used in Step 1 is triethylamine. In some embodiments, the base used in Step 1 is Na₂CO₃. In some embodiments, the base used in Step 1 is K₂CO₃. In some embodiments, about 1, 2, 3, 4, 5, or 6 equivalents of the base is used in Step 1.

In some embodiments, the suitable solvent used in Step 1 is a single solvent. In some embodiments, the suitable solvent used in Step 1 is a cosolvent mixture. In some embodiments, the suitable solvent used in Step 1 is acetonitrile, dimethylformamide, dimethoxyethane, 2-methyltetrahydrofuran, methyl tert-butyl ether, cyclopentyl methyl ether, tetrahydrofuran, diisopropyl ether, 1,4-dioxane, toluene, water, or a combination thereof. In some embodiments, the suitable solvent used in Step 1 is toluene.

In some embodiments, the temperature used in Step 1 is between about 40° and 120° C., preferably between about 50° C. and 100° C. In some embodiments, the temperature used in Step 1 is about 60° C. In some embodiments, the temperature used in Step 1 is about 80° C. In some embodiments, the temperature used in Step 1 is about 90° C. In some embodiments, the temperature used in Step 1 is between 75° C. and 85° C.

In some embodiments, the B group of Compound 7 is a boronic acid or a boronic ester. In some embodiments, B is

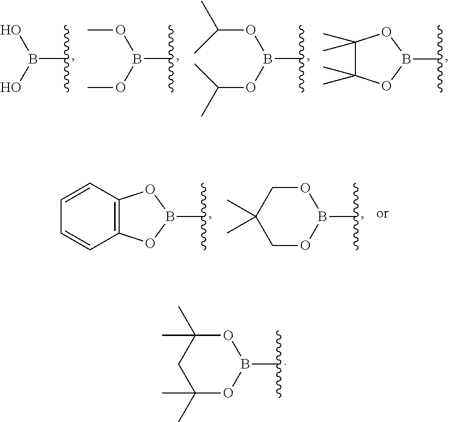

In some embodiments, B is a boronic acid. In some embodiments, B is

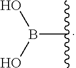 [SMILES: *B(O)O]

In some embodiments, B is a boronic ester. In some embodiments, B is

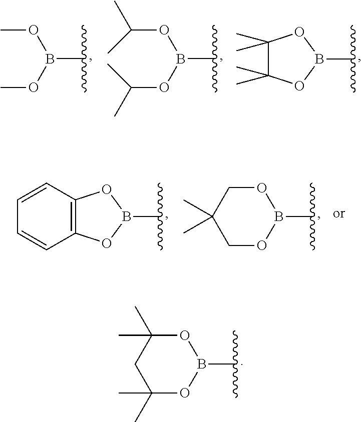

In some embodiments, B is

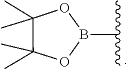 [SMILES: *B1OC(C)(C)C(C)(C)O1]

In some embodiments, B is a trifluoroborate. In some embodiments, B is

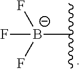 [SMILES: *[B-](F)(F)F]

In some embodiments, the X′ group of Compound 7 is a halogen. In some embodiments, X′ is Cl, Br, or I. In some embodiments, X′ is Br or I. In some embodiments, X′ is Br. In some embodiments, X′ is I.

In some embodiments, Compound 7 is Compound 7a:

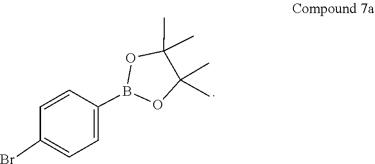 [SMILES: CC1(C)OB(c2ccc(Br)cc2)OC1(C)C]

In some embodiments, the X group of Compound 4 is a halogen. In some embodiments, X is Cl, Br, or I. In some embodiments, X is Br or I. In some embodiments, X is Br. In some embodiments, X is I.

In some embodiments, the R group of Compound 4 is C₁-C₂₀ alkyl, C₁-C₂₀ alkenyl, C₃-C₁₀ cycloalkyl, or C₃-C₁₀ cycloalkenyl. In some embodiments, R is C₁-C₂₀ alkyl or C₁-C₂₀ alkenyl. In some embodiments, R is methyl, ethyl, propyl, isopropyl, butyl, isobutyl, tert-butyl, isoamyl, pentyl, hexyl, heptyl, octyl, nonyl, terpenyl, bornyl, allyl, linalyl or geranyl. In some embodiments, R is C₁-C₁₀ alkyl. In some embodiments, R is C₁-C₆alkyl. In some embodiments, R is C₁-C₄ alkyl. In some embodiments, R is methyl, ethyl, propyl, isopropyl, butyl, isobutyl, tert-butyl, isoamyl, pentyl, or hexyl. In some embodiments, R is methyl or ethyl. In some embodiments, R is methyl. In some embodiments, R is ethyl.

In some embodiments, Compound 4 is Compound 4a, Compound 4b, Compound 4c, or Compound 4d:

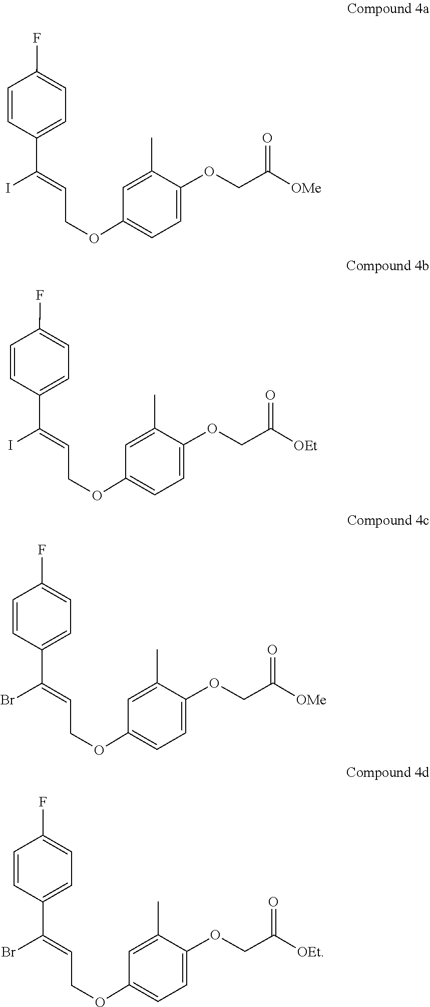 [SMILES: COC(=O)COc1ccc(OC/C=C(\I)c2ccc(F)cc2)cc1C ; CCOC(=O)COc1ccc(OC/C=C(\I)c2ccc(F)cc2)cc1C ; COC(=O)COc1ccc(OC/C=C(\Br)c2ccc(F)cc2)cc1C ; CCOC(=O)COc1ccc(OC/C=C(\Br)c2ccc(F)cc2)cc1C]

In some embodiments, the R group of Compound 8 is C₁-C₂₀ alkyl, C₁-C₂₀ alkenyl, C₃-C₁₀ cycloalkyl, or C₃-C₁₀ cycloalkenyl. In some embodiments, R is C₁-C₂₀ alkyl or C₁-C₂₀ alkenyl. In some embodiments, R is methyl, ethyl, propyl, isopropyl, butyl, isobutyl, tert-butyl, isoamyl, pentyl, hexyl, heptyl, octyl, nonyl, terpenyl, bornyl, allyl, linalyl or geranyl. In some embodiments, R is C₁-C₂₀ alkyl. In some embodiments, R is C₁-C₁₀ alkyl. In some embodiments, R is C₁-C₆ alkyl. In some embodiments, R is methyl, ethyl, propyl, isopropyl, butyl, isobutyl, tert-butyl, isoamyl, pentyl, or hexyl. In some embodiments, R is methyl or ethyl. In some embodiments, R is methyl. In some embodiments, R is ethyl.

In some embodiments, the X group of Compound 8 is a halogen. In some embodiments, X is Cl, Br, or I. In some embodiments, X is Br or I. In some embodiments, X is Br. In some embodiments, X is I.

In some embodiments, Compound 8 is Compound 8a, Compound 8b, Compound 8c, or Compound 8d:

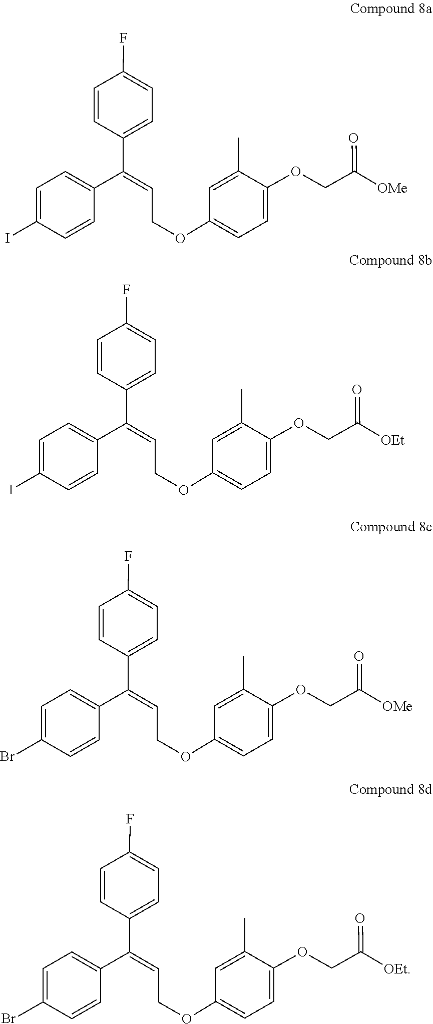 [SMILES: COC(=O)COc1ccc(OC/C=C(/c2ccc(F)cc2)c2ccc(I)cc2)cc1C ; CCOC(=O)COc1ccc(OC/C=C(/c2ccc(F)cc2)c2ccc(I)cc2)cc1C ; COC(=O)COc1ccc(OC/C=C(/c2ccc(F)cc2)c2ccc(Br)cc2)cc1C ; CCOC(=O)COc1ccc(OC/C=C(/c2ccc(F)cc2)c2ccc(Br)cc2)cc1C]

### Step 2: Synthesis of the Compound 5

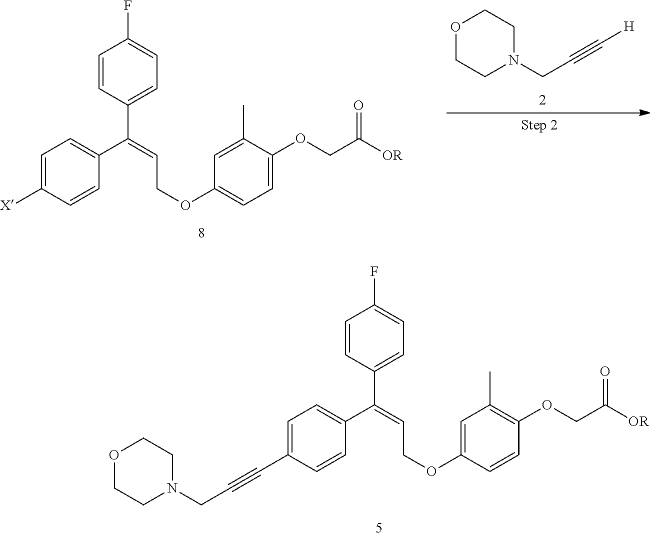

As disclosed herein, Compound 5, or salt thereof, is prepared from Compound 8 and Compound 2, or salt thereof. In some embodiments, Compound 5, or salt thereof, is produced by a Sonogashira cross-coupling of Compound 8 and Compound 2, or a salt thereof. In some embodiments, Compound 8 is reacted with Compound 2, or salt thereof, in the presence of a coupling catalyst, a suitable copper(I) cocatalyst, a suitable base, and in a suitable solvent to yield Compound 5, or salt thereof.

In some embodiments, the coupling catalyst in Step 2 is a palladium catalyst. In some embodiments, the palladium catalyst is a palladium(0) catalyst. In other embodiments, the palladium catalyst is a palladium(II) catalyst. In some embodiments, the palladium catalyst is precoordinated with a ligand. In some embodiments, Step 2 further comprises adding an exogenous ligand. In some embodiments, the ligand is a phosphine ligand. In some embodiments, the ligand is an aliphatic phosphine ligand, such as trimethyl phosphine, tricyclohexylphosphine, tri-tert-butyl-phosphine or the like. In some embodiments, the ligand is an aromatic phosphine, such as XPhos, SPhos, JohnPhos, Amphos, triphenylphosphine, methyldiphenylphosphine, or the like. In some embodiments, the ligand is a phosphite ligand, such as trimethylphosphite, triphenylphosphite, or the like. In some embodiments, the ligand is a bis-phosphine ligand, such as diphenylphosphinomethane (dppm), diphenyl phosphinoethane (dppe), 1,1′-bis(diphenylphosphino)ferrocene (dppf), or the like. In some embodiments, the ligand is triphenylphosphine. In some embodiments, the palladium catalyst is Pd(PPh₃)₂Cl₂. In some embodiments, the palladium catalyst is Pd(PPh₃)₃Cl. In some embodiments, the palladium catalyst is Pd(PPh₃)₄. In some embodiments, the amount of palladium used in Step 2 is from about 0.005 equiv to about 0.1 equiv. In some embodiments, the amount of palladium used in Step 2 is about 0.005, about 0.01, about 0.02, about 0.03, about 0.04, about 0.05, about 0.06, about 0.07, about 0.08, about 0.09, or about 0.1 equiv. In some embodiments, the amount of palladium used in Step 2 is about 0.01 equiv.

In some embodiments, the copper(I) cocatalyst in Step 2 is a copper(I) salt. In some embodiments, the copper(I) cocatalyst in Step 2 is CuCl, CuBr, or CuI. In some embodiments, the copper(I) cocatalyst is CuI. In some embodiments, the copper(I) cocatalyst is a copper(I)-N-heterocyclic carbene (Copper-NHC) complex. In some embodiments, the amount of copper(I) cocatalyst used in Step 2 is from about 0.001 equiv to about 0.1 equiv. In some embodiments, the amount of copper(I) cocatalyst used in Step 2 is about 0.001, about 0.002, about 0.003, about 0.004, about 0.005, about 0.01, about 0.02, about 0.03, about 0.04, about 0.05, about 0.06, about 0.07, about 0.08, about 0.09, or about 0.1 equiv. In some embodiments, the amount of copper(I) cocatalyst used in Step 2 is about 0.005 equiv.

In some embodiments, suitable bases in Sonogashira reactions include amine bases. In some embodiments, suitable amine bases for Sonogashira reactions are tertiary amine bases. Suitable amine bases for Sonogashira reactions include, but are not limited to, triethylamine, diisopropylethylamine, 1,2,2,6,6-pentamethylpiperidine, tributylamine, 1,8-diazabicycloundec-7-ene (DBU), or the like. In some embodiments, the base used in Step 2 is triethylamine. In some embodiments, the base used in Step 2 is 1,8-diazabicycloundec-7-ene (DBU). In some embodiments, about 1, about 2, about 3, about 4, about 5, or about 6 equivalents of the base is used in Step 2.

In some embodiments, the solvent system used in Step 2 is a single solvent. In some embodiments, the solvent system used in Step 2 is a cosolvent mixture. In some embodiments, the solvent system used in Step 2 is acetonitrile, dimethylformamide, diethyl ether, ethanol, tetrahydrofuran, 2-methyltetrahydrofuran, isopropyl alcohol, 1,4-dioxane, toluene, water, or a combination thereof. In some embodiments, the solvent system used in Step 2 is toluene.

In some embodiments, the temperature used in Step 2 is between about 40° and about 100° C., preferably between about 50° C. and about 70° C. In some embodiments, the temperature used in Step 2 is between 65° C. and about 75° C.

In some embodiments, the free base form of Compound 2 is used. In some embodiments, a salt form of Compound 2 is used. In some embodiments, an acid addition salt form of Compound 2 is used. In some embodiments Compound 2 is used as a hydrochloride salt form. In some embodiments, Compound 2, or salt thereof, is the hydrochloride salt Compound 2a:

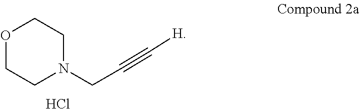

In some embodiments, Compound 5, or salt thereof, is used as the free base form of Compound 5. In some embodiments, Compound 5, or salt thereof, is used as the acid addition salt form of Compound 5. In some embodiments, Compound 5, or salt thereof, is used as the hydrochloride salt.

In some embodiments, the R group of Compound 5, or salt thereof, is C₁-C₂₀ alkyl, C₁-C₂₀ alkenyl, C₃-C₁₀ cycloalkyl, or C₃-C₁₀ cycloalkenyl. In some embodiments, R is C₁-C₂₀ alkyl or C₁-C₂₀ alkenyl. In some embodiments, R is methyl, ethyl, propyl, isopropyl, butyl, isobutyl, tert-butyl, isoamyl, pentyl, hexyl, heptyl, octyl, nonyl, terpenyl, bornyl, allyl, linalyl or geranyl. In some embodiments, R is C₁-C₂₀ alkyl. In some embodiments, R is C₁-C₁₀ alkyl. In some embodiments, R is C₁-C₆ alkyl. In some embodiments, R is methyl, ethyl, propyl, isopropyl, butyl, isobutyl, tert-butyl, isoamyl, pentyl, or hexyl. In some embodiments, R is methyl or ethyl. In some embodiments, R is methyl. In some embodiments, R is ethyl.

In some embodiments, Compound 5, or salt thereof, is Compound 5a, or salt thereof, Compound 5b, or salt thereof, the hydrochloride salt Compound 5c, or the hydrochloride salt Compound 5d:

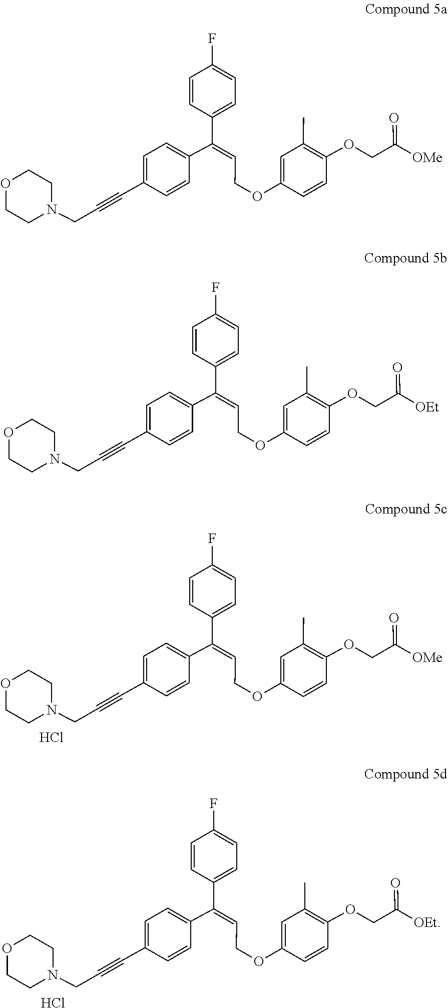 [SMILES: COC(=O)COc1ccc(OC/C=C(/c2ccc(F)cc2)c2ccc(C#CCN3CCOCC3)cc2)cc1C ; CCOC(=O)COc1ccc(OC/C=C(/c2ccc(F)cc2)c2ccc(C#CCN3CCOCC3)cc2)cc1C ; COC(=O)COc1ccc(OC/C=C(/c2ccc(F)cc2)c2ccc(C#CCN3CCOCC3)cc2)cc1C ; Cl ; CCOC(=O)COc1ccc(OC/C=C(/c2ccc(F)cc2)c2ccc(C#CCN3CCOCC3)cc2)cc1C ; Cl]

Due to the fact that the synthetic methods described above utilize a transition metal catalyst, purification steps are performed to reduce the amount of palladium in the product. Purification steps to reduce the amount of palladium in a product are conducted so that active pharmaceutical ingredients meet palladium specification guidelines. (“Guideline on the Specification Limits for Residues of Metal Catalysts” European Medicines Agency Pre-authorisation Evaluation of Medicines for Human Use, London, January 2007, Doc. Ref. CPMP/SWP/QWP/4446/00 corr.). In some embodiments, purification steps to reduce the amount of palladium in a product includes, but is not limited to, treatment with solid trimercaptotriazine (TMT), polystyrene-bound TMT, mercapto-porous polystyrene-bound TMT, polystyrene-bound ethylenediamine, activated carbon, glass bead sponges, Smopex™, silica bound scavengers, thiol-derivatized silica gel, N-acetylcysteine, n-Bu₃P, crystallization, extraction, L-cysteine, n-Bu₃P/lactic acid (Garrett et al., Adv. Synth. Catal. 2004, 346, 889-900). In some embodiments, activated carbon includes but is not limited to DARCO® KB-G, DARCO® KB-WJ. In one aspect silica bound scavengers include but are not limited to

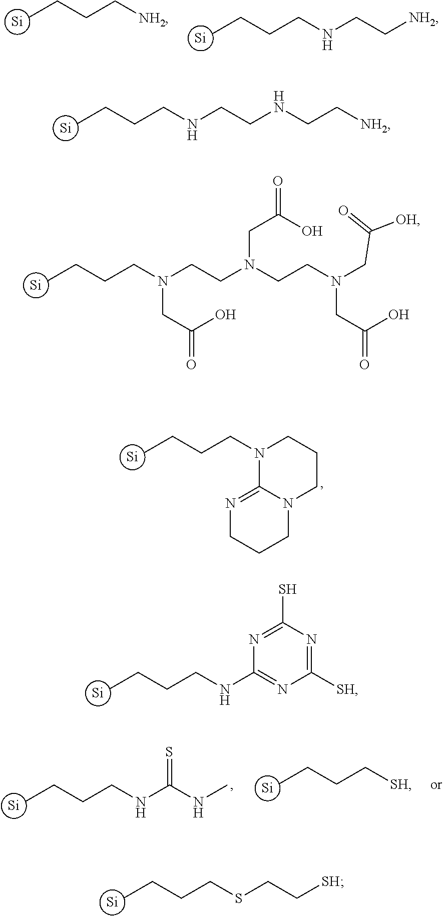

where

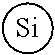

denotes silica gel. In some embodiments, the purification steps to reduce the amount of palladium include the use of activated carbon, derivatized silica gel (e.g., thiol derivatized silica gel), or combinations thereof.

In some embodiments, Compound 5, or salt thereof, is further treated with a metal scavenger to remove residual palladium. In some embodiments, the metal scavenger comprises SiO₂, charcoal, aqueous solution of L-cysteine, a Silicycle metal scavenger, Si-thiol, SiliaBond DMT, SiliaBond Cysteine, or 3-mercaptopropyl ethyl sulfide silica. In some embodiments, the scavenger loading (w/w) is about 1:3, about 1:2, or about 1:1. In some embodiments, the metal scavenger is 3-mercaptopropyl ethyl sulfide silica.

In some of these embodiments, palladium levels are reduced to about 10 ppm. In some of these embodiments, palladium levels are reduced sufficiently to be undetectable.

In some embodiments, the presence of residual heavy metal (e.g., palladium) impurities is determined by utilizing methods known in the art. In some embodiments, the presence of residual heavy metal (e.g., palladium) impurities is determined by the use of inductively coupled plasma mass spectrometry (ICP-MS). In some embodiments, the presence of residual heavy metal (e.g., palladium) impurities is determined by the use of techniques described in U.S. Pharmacopeia General Chapter <231> Heavy Metals.

In some embodiments, the final two steps of the synthesis follow the same steps as described above for Scheme A.

### Step 3: Synthesis of Compound I

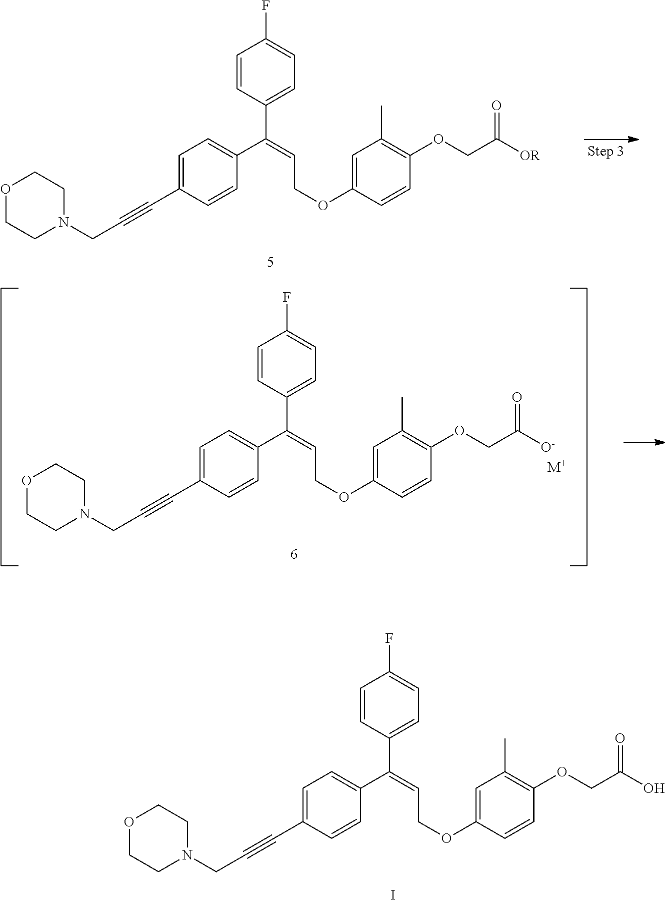

As disclosed herein, Compound 6 is prepared from Compound 5, or salt thereof. In some embodiments, saponification of Compound 5, or salt thereof, in Step 3, followed by acid neutralization, yields the carboxylic acid Compound I. In some embodiments, Compound 5, or salt thereof, is reacted with sodium hydroxide, potassium hydroxide or lithium hydroxide in a suitable solvent to yield Compound 6. In some embodiments, treatment of Compound 6 with a suitable acid in a suitable solvent provides Compound I. In some embodiments, Compound 6 is not isolated before treatment with the suitable acid in the suitable solvent.

In some embodiments, Compound 5, or salt thereof, is reacted with sodium hydroxide to provide Compound 6, wherein M⁺ is Na⁺ (i.e., Compound II). In some embodiments, Compound 5, or salt thereof, is reacted with potassium hydroxide to provide Compound 6, wherein M⁺ is K⁺. In some embodiments, Compound 5, or salt thereof, is reacted with lithium hydroxide to provide Compound 6, wherein M⁺ is Li⁺. In some embodiments, about 1, about 1.5, about 2, about 2.5, about 3, about 4, or about 5 equivalents of sodium hydroxide, potassium hydroxide or lithium hydroxide is used in Step 3. In some embodiments, about 2.5 equivalents of sodium hydroxide are used in Step 3.

In some embodiments, the suitable solvent used in Step 3 is a single solvent. In some embodiments, the suitable solvent used in Step 3 is a cosolvent mixture. In some embodiments, the suitable solvent used in Step 3 is water, methanol, ethanol, tetrahydrofuran, ethyl acetate, or a combination thereof. In some embodiments, the suitable solvent used in Step 3 is a mixture of ethanol and water.

In some embodiments, the temperature used in Step 3 is between about 0° and 50° C., preferably between about 15° C. and 30° C. In some embodiments, the temperature used in Step 3 is about 25° C. In some embodiments, the temperature used in Step 3 is between 15° C. and 25° C.

In some embodiments, the suitable acid for neutralization in Step 3 is acetic acid, citric acid, oxalic acid, lactic acid, hydrochloric acid, nitric acid, or sulfuric acid. In some embodiments, the suitable acid is acetic acid.

In some embodiments, the suitable solvent used in the neutralization step of Step 3 is a single solvent. In some embodiments, the suitable solvent is a cosolvent mixture. In some embodiments, the suitable solvent is water, methanol, ethanol, tetrahydrofuran, ethyl acetate, or a combination thereof. In some embodiments, the suitable solvent is water.

### Step 4: Synthesis of Compound II

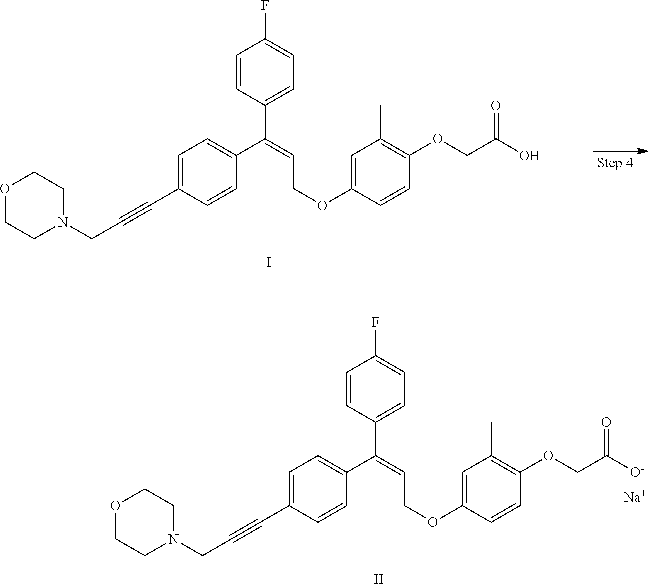

As disclosed herein, Compound II is prepared from Compound I. In some embodiments, Compound I is treated with a sodium solution to yield Compound II. In some embodiments, Compound I is treated with a sodium hydroxide solution in the presence of a suitable solvent to provide Compound II.

In some embodiments, the suitable solvent used in Step 4 is a single solvent. In some embodiments, the suitable solvent is a cosolvent mixture. In some embodiments, the suitable solvent is water, methanol, ethanol, tetrahydrofuran, ethyl acetate, acetonitrile, acetone, or a combination thereof. In some embodiments, the suitable solvent is water, ethyl acetate, acetonitrile, acetone, or a combination thereof. In some embodiments, the suitable solvent is a mixture of water and ethyl acetate.

In some embodiments, Step 4 is performed at room temperature. In some embodiments, Step 4 is performed at or above room temperature. In some embodiments, the temperature used in Step 4 is between about 20° and 60° C. In some embodiments, the temperature used in Step 4 is about 40° C. In some embodiments, the temperature used in Step 4 is about 50° C. In some embodiments, Step 4 is performed below room temperature.

In some embodiments, samples of Compound I and/or Compound II include a detectable amount of one or more impurities. In some embodiments, these impurities are undesired compounds produced during the synthesis of Compound I and/or Compound II. In some embodiments, the synthetic procedures described herein provide for samples of Compound I and/or Compound II that are substantially free of synthetic impurities.

Described herein is Compound II substantially free of sodium (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate. In some embodiments, the amount of sodium (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate is less than 1% (w/w). In some embodiments, the amount of sodium (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate is less than 0.5% (w/w). In some embodiments, the amount of sodium (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate is less than 0.15% (w/w). In some embodiments, the amount of sodium (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate is less than 0.10% (w/w). In some embodiments, the amount of sodium (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate in undetectable. In some embodiments, the amount of sodium (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate in undetectable by NMR, HPLC, or the like.

Also described herein is the compound methyl (E)-2-(4-((3-(4-fluorophenyl)-3-(4-(3-morpholinoprop-1-yn-1-yl)phenyl)allyl)oxy)-2-methylphenoxy)acetate substantially free of methyl (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate. In some embodiments, the amount of methyl (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate is less than 1% (w/w). In some embodiments, the amount of methyl (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate is less than 0.5% (w/w). In some embodiments, the amount of methyl (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate is less than 0.15% (w/w). In some embodiments, the amount of methyl (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate is less than 0.10% (w/w). In some embodiments, the amount of methyl (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate in undetectable. In some embodiments, the amount of methyl (E)-2-(4-((3-(4-fluorophenyl)-3-(4′-(3-morpholinoprop-1-yn-1-yl)-[1,1′-biphenyl]-4-yl)allyl)oxy)-2-methylphenoxy)acetate in undetectable by NMR, HPLC, or the like.

In some embodiments, compounds and solid state forms described herein are synthesized as outlined in the Examples.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound disclosed herein with an acid. In some embodiments, the compound disclosed herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound disclosed herein is prepared as a hydrochloride salt.

In some embodiments, pharmaceutically acceptable salts are obtained by reacting a compound disclosed herein with a base. In some embodiments, the compound disclosed herein is acidic and is reacted with a base. In such situations, an acidic proton of the compound disclosed herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

In some embodiments, a compound disclosed herein is prepared as the sodium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

Residual solvents in active pharmaceutical ingredients (APIs) originate from the manufacture of API. In some cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound II, comprise an organic solvent(s). In some embodiments, compositions comprising Compound II include a residual amount of an organic solvent(s). In some embodiments, compositions comprising Compound II comprise a residual amount of a Class 3 solvent. In some embodiments, the Class 3 solvent is selected from the group consisting of acetic acid, acetone, anisole, 1-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. In some embodiments, the Class 3 solvent is selected from ethyl acetate, isopropyl acetate, tert-butylmethylether, heptane, isopropanol, and ethanol.

In some embodiments, the compositions comprising Compound II include a detectable amount of an organic solvent. In some embodiments, the organic solvent is a Class 3 solvent.

In other embodiments are compositions comprising Compound II wherein the composition comprises a detectable amount of solvent that is less than about 1%, wherein the solvent is selected from acetone, 1,2-dimethoxyethane, acetonitrile, ethyl acetate, tetrahydrofuran, methanol, ethanol, heptane, and 2-propanol. In a further embodiment are compositions comprising Compound II wherein the composition comprises a detectable amount of solvent which is less than about 5000 ppm. In yet a further embodiment are compositions comprising Compound II, wherein the detectable amount of solvent is less than about 5000 ppm, less than about 4000 ppm, less than about 3000 ppm, less than about 2000 ppm, less than about 1000 ppm, less than about 500 ppm, or less than about 100 ppm.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or altered metabolic pathways to reduce undesirable metabolites or reduced dosage requirements.

In some embodiments, one or more hydrogen atoms on Compound II are replaced with deuterium. In some embodiments, substitution with deuterium affords certain therapeutic advantages resulting from greater metabolic stability, such as, for example, increased in vivo half-life or reduced dosage requirements.

In one aspect, described is a compound with the following structure:

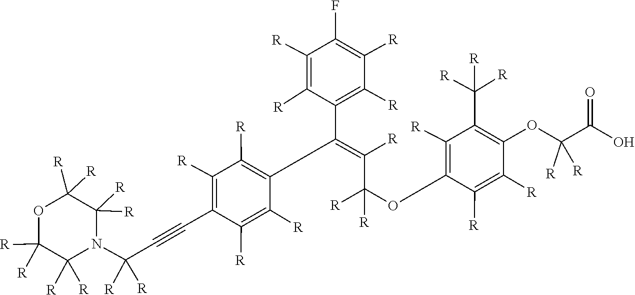

wherein,
each R is independently selected from hydrogen or deuterium,
or a pharmaceutically acceptable salt thereof.

In some embodiments, the pharmaceutically acceptable salt of the compound is a sodium salt.

The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “halo” or, alternatively, “halogen” or “halide” means fluoro, chloro, bromo or iodo. In some embodiments, halo is fluoro, chloro, or bromo.

The term “moiety” refers to a specific segment or functional group of a molecule. Chemical moieties are often recognized chemical entities embedded in or appended to a molecule.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes.

The term “subject” or “patient” encompasses mammals. Examples of mammals include, but are not limited to, any member of the Mammalian class: humans, non-human primates such as chimpanzees, and other apes and monkey species. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action.

### Methods of Treatment

In one embodiment, the compounds disclosed herein, or a pharmaceutically acceptable salt thereof, are used in the preparation of medicaments for the treatment of diseases or conditions in a mammal that would benefit from modulation of PPARδ activity. Methods for treating any of the diseases or conditions described herein in a mammal in need of such treatment, involves administration of pharmaceutical compositions that include at least one compound disclosed herein or a pharmaceutically acceptable salt, active metabolite, prodrug, or pharmaceutically acceptable solvate thereof, in therapeutically effective amounts to said mammal.

In some embodiments, Compound I, or a pharmaceutically acceptable salt thereof (e.g. Compound II) is used in the treatment of a kidney disease in a mammal. In some embodiments, the kidney disease is Alport syndrome, Goodpasture syndrome, thin basement membrane nephropathy (TBMN), focal segmental glomerulosclerosis (FSGS), benign familial hematuria (BFH), post-transplant anti-GBM (Glomerular Basement Membrane) nephritis. In some embodiments, the kidney disease is X-linked Alport syndrome (XLAS), autosomal recessive Alport syndrome (ARAS) or autosomal dominant Alport syndrome (ADAS).

In some embodiments, Compound I, or a pharmaceutically acceptable salt thereof (e.g. Compound II) is used in the treatment of muscle atrophy in a mammal. In some embodiments, the muscle atrophy is secondary to a chronic disease. In some embodiments, the chronic disease is multiple sclerosis, amyotrophic lateral sclerosis, spinal muscular atrophy, critical illness neuropathy, cancer, congestive heart failure, chronic pulmonary disease, chronic renal failure, chronic liver disease, diabetes mellitus, Cushing syndrome, chronic infection, glucorticoid-induced myopathy, statin-induced myopathy, polymyositis or dermatomyositis. In some embodiments, the chronic disease is a neurologic disease or drug-induced muscle disease. In some embodiments, the muscle atrophy is secondary to a genetic disease that primarily affect skeletal muscle. In some embodiments, the genetic disease is muscular dystrophy or myotonic dystrophy. In some embodiments, the muscle atrophy results from a muscle disease. In some embodiments, the muscle disease is muscular dystrophy, polymyositis, or myotonia. In some embodiments, the muscle disease occurs as a response to systemic illness. In some embodiments, the systemic illness is hypothyroidism, hyperthyroidism, adrenal gland depletion, diabetes mellitus, or an autoimmune disease. In some embodiments, the systemic illness is cancer, Acquired Immune Deficiency Syndrome (AIDS), chronic obstructive lung disease, congestive heart failure, cardiomyopathy, chronic liver disease, renal disease, emphysema, tuberculosis, osteomalacia, hormonal deficiency, anorexia nervosa, and generalized malnutrition.

In some embodiments, Compound I, or a pharmaceutically acceptable salt thereof (e.g. Compound II) is used in the treatment of a primary mitochondrial myopathy in a mammal. In some embodiments, the mammal has been diagnosed with Kearns-Sayre syndrome (KSS), Leigh syndrome, maternally inherited Leigh syndrome (MILS), Mitochondrial DNA depletion syndrome (MDS), Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy ataxia and retinitis pigmentosa (NARP), Pearson syndrome, or Progressive external ophthalmoplegia (PEO).

In some embodiments, Compound I, or a pharmaceutically acceptable salt thereof (e.g. Compound II) is used in the treatment of a fatty acid oxidation disorder (FAOD) in a mammal. In some embodiments, the fatty acid oxidation disorder (FAOD) comprises carnitine transporter deficiency, carnitine/acylcarnitine translocase deficiency, carnitine palmitoyl transferase deficiency Type 1, carnitine palmitoyl transferase deficiency Type 2, glutaric acidemia Type 2, long-chain 3-hydroxyacyl CoA dehydrogenase deficiency, medium-chain acyl CoA dehydrogenase deficiency, short-chain acyl CoA dehydrogenase deficiency, short-chain 3-hydroxyacyl CoA dehydrogenase deficiency, trifunctional protein deficiency, or very long-chain acyl CoA dehydrogenase deficiency, or a combination thereof. In some embodiments, the fatty acid oxidation disorder comprises carnitine palmitoyltransferase II (CPT2) deficiency, very long-chain Acyl-CoA dehydrogenase (VLCAD) deficiency, long-chain 3-hydroxyacyl-CoA dehydrogenase (LCHAD) deficiency, Trifunctional Protein (TFP) Deficiency; or a combination thereof.

### EXAMPLES

The following examples are provided for illustrative purposes only and not to limit the scope of the claims provided herein.

### Example 1: Preparation of methyl (Z)-2-(4-((3-(4-fluorophenyl)-3-iodoallyl)oxy)-2-methylphenoxy)acetate (Compound 4a) and methyl (Z)-2-(4-((3-bromo-3-(4-fluorophenyl)allyl)oxy)-2-methylphenoxy)acetate (Compound 4c)

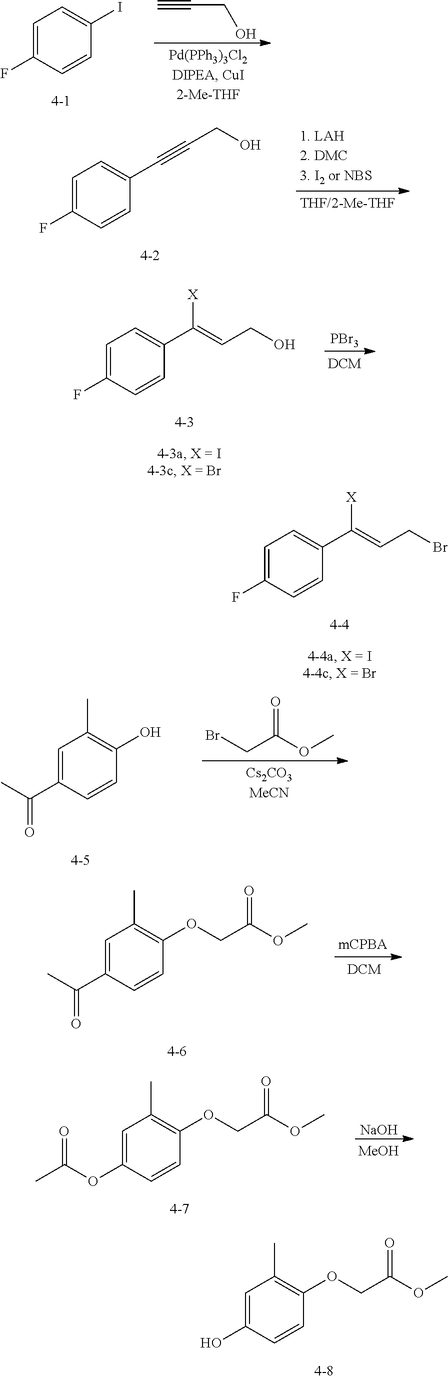

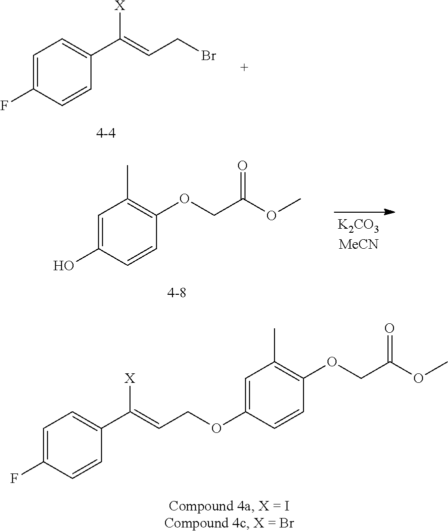

### Example 1-1: Preparation of 3-(4-fluorophenyl)prop-2-yn-1-ol (Compound 4-2)

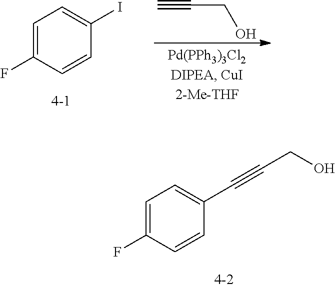

A 100 L jacketed reactor was charged with 36 L of 2-Me-THF and 4-fluoroiodobenzene (6.0 kg, 27 mol) and promptly degassed. In a nitrogen atmosphere, N,N-diisopropylethylamine (7 L), copper(I) iodide (200 g, 1.05 mol), and Pd(PPh₃)₃Cl (91 g, 85 mmol) were added into the reactor. After the jacket temperature reached 20° C., propargylalcohol (1.9 L, 32.4 mol) was added dropwise over a period of 2 h while keeping the reaction temperature in the range of 30-40° C. After the addition, the reaction mixture was kept at 20° C. for 30 minutes and a full conversion was observed by LC/MS analysis. 1M hydrochloric acid (20 L) was added quickly and the pH of the reaction mixture was 5˜7. After stirring at 30° C. for 30 minutes, the layers were separated and the lower aqueous layer was drained out. 20 L of water were added to the reactor and the mixture was stirred at rt for 30 minutes. After separation of layers, the lower layer was drained out, and 12 L of 6% sodium bicarbonate aqueous solution was added to the reactor. After stirring at rt for 30 minutes, the lower layer was drained out and the organic phase was washed with 20 L of brine. After separation of the aqueous layer, the organic phase was collected and the reactor was washed with 2-Me-THF. The combined organic phase was concentrated under reduced pressure and 10.88 kg crude product was obtained.

Silica gel (12 kg) was loaded into a 30 L column and conditioned with hexanes. The crude product was loaded on top of the column. The product was eluted with ethyl acetate:hexanes. Fractions containing the pure product were pooled and concentrated under reduced pressure to give the desired product, which was stored in a freezer. ¹H-NMR (300 MHz, CDCl₃): δ 7.45-7.40 (m, 2H), 7.04-6.98 (m, 2H), 4.49 (s, 2H), 1.96 (s, 1H).

### Alternative Conditions

To a reaction vessel containing Compound 4-1 (1 eq) in 2-Me-THF (6 ml/g), stirring at 15-25° C. under N₂ atmosphere, was added DIPEA (1 eq), CuI (0.04 eq) and Pd(PPh₃)₂Cl₂ (0.005 eq). The temperature was adjusted to 30-40° C. and propargyl alcohol (1.2 eq) was added dropwise. The resulting mixture was stirred at 30-40° C. for 5-10 h. The reaction was monitored, stirring at 30-40° C. until propargyl alcohol ≤100 ppm, and then cooled to 15-25° C. The reaction mixture was then filtered, and the residue washed with 2-Me-THF (2 ml/g). The filtrate was adjusted to pH 5-7 with 1M HCl (2-5 ml/g) at 10-20° C. The mixture was stirred at 15-25° C. for 30-60 minutes, then allowed to stand at 15-25° C. for 30-60 minutes. The organic phase was separated and stirred with 7% NaHCO₃ solution (2 ml/g) at 15-25° C. for 30-60 mins, filtered, then allowed to stand at 15-25° C. for 30-60 mins. Again, the organic phase was separated and stirred with 7% NaHCO₃ solution (2 ml/g) at 15-25° C. for 30-60 mins, filtered, then allowed to stand at 15-25° C. for 30-60 minutes. The organic phase was again separated and stirred with 10% Na₂SO₄ (3 ml/g) at 15-25° C. for 30-60 minutes then allowed to stand at 15-25° C. for 30-60 minutes. The organic layer was concentrated below 45° C. to 2.5-3.5 ml/g. Heptane was added (9-12 ml/g) to the separated aqueous phase and the mixture stirred at 15-25° C. for 30-60 mins, then filtered through silica gel. The residue was washed with heptane/2-Me-THF (9:1, 10-20 ml/g), and both filtrates were combined with the first concentrated organic layer. The organic layer was separated, filtered through silica gel, and the residue was washed with heptane/2-Me-THF (1:1, 30-35 ml/g). Both filtrates were combined and concentrated below 45° C. to 3-5 ml/g. Next, 2-MeTHF (5 ml/g) was added and the mixture was again concentrated below 45° C. to 3-5 ml/g. This operation was repeated and if Karl Fischer (KF) analysis of the resulting mixture was >0.5%, further 2-Me-THF was added and the mixture was again concentrated to 6-8 ml/g until KF ≤0.5%.

### Example 1-2: Preparation of (Z)-3-(4-fluorophenyl)-3-iodoprop-2-en-1-ol (Compound 4-3a)

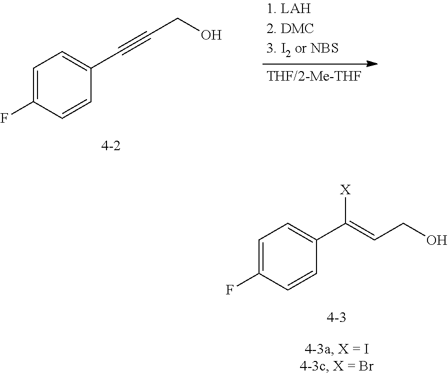

To solution of 2-Me-THF (6.8 L) was charged lithium aluminum hydride (287 g, 7.55 mol) in portion under the flush of nitrogen. After addition, the contents were cooled to 0° C. A solution of 3-(4-fluorophenyl)prop-2-yn-1-ol (4-2, 800 g, 5.33 mol) in 2-Me-THF (2 L) was added dropwise over 60 minutes while keeping the reaction temperature below −5° C. After addition, the reaction mixture was stirred at −5° C. for 60 minutes and reached a full conversion. A solution of dimethyl carbonate (DMC, 624 mL, 6.4 mol) in 2-Me-THF (1.6 L) was added dropwise while keeping the reaction temperature below 0° C. Toward the end of the addition, the temperature begins to drop quickly and the remaining carbonate solution was added over 5 minutes. The mixture was stirred for 30 minutes and then cooled to −10° C. A solution of iodine (1.62 kg, 6.4 mol) in anhydrous 2-Me-THF (2.0 L) was added to the mixture dropwise while keeping the temperature below 0° C. The resulting mixture was stirred overnight and the temperature was allowed warm to room temperature slowly.

Sodium sulfite solution (0.86 M, 5 L) was added dropwise to quench the reaction. The temperature was increased slightly throughout the addition (20-35° C.). During the addition, the mixture became a yellow gel and the stirring became difficult. The addition of sodium sulfite solution was continued and the most of the gel was broken up into a yellow liquid.

The procedure of other three batches was completed as described as above. And those four batches were combined for work-up. The combined quenched mixture was stirred for 30 minutes. Then upper layer (organic layer) was separated. To the lower layer (aqueous emulsion layer) was added hydrochloric acid (3 M, 50 L). After stirred for 30 minutes, the emulsion was broken up. This mixture was then extracted with ethyl acetate (20 L), and aqueous layer was removed. The organic layers were combined and washed with 15% NaCl/5% Na₂HPO₄ solution (20 L), the phases were allowed to separate and the lower phase was removed. The pH was checked and adjusted to be in the neutral range (6-8). The organic layer was dried over Na₂SO₄, filtered and concentrated to give 5.6 kg of the crude product, which was protected from light. ¹H-NMR (300 MHz, CDCl₃): δ 7.48-7.43 (m, 2H), 7.05-6.98 (m, 2H), 6.22-6.18 (m, 1H), 4.38 (d, J=5.7 Hz, 2H), 2.19 (s, 1H).

### Example 1-3: Preparation of (Z)-3-bromo-3-(4-fluorophenyl)prop-2-en-1-ol (Compound 4-3c)

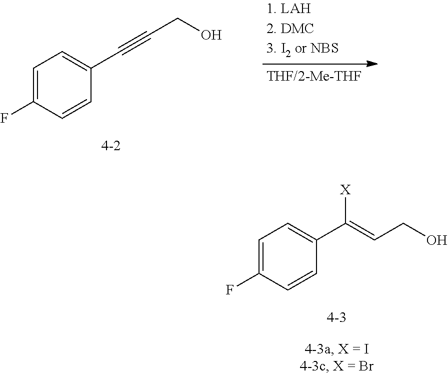

The synthesis of Compound 4-3c begins with Compound 4-2, the synthesis of which can be found in Example 1-1 above. Solid lithium aluminum hydride (1.1 eq) was charged to a mix of anhydrous THF (3 ml/g) and anhydrous 2-Me-THF (4.6 ml/g) and stirred at 10-30° C. for 2-6 h and then cooled to between −15 and −5° C. The solution of 3-(4-fluorophenyl)prop-2-yn-1-ol (4-2) in 2-MeTHF (1 eq) was added dropwise and the mixture was stirred at between −15 and −5° C. for 3-5 h. The reaction was monitored by IPC and further additions of LAH were made as required. Following completion of reaction, a solution of dimethyl carbonate (DMC) in 2-MeTHF (1.2 eq) was added dropwise keeping the temperature between −15 and −5° C. The mixture was stirred at between −15 and −5° C. for 1-2 h and then cooled to between −40 and −20° C. NBS (1.02 eq) was added and the reaction mixture stirred at between −40 and −20° C. for 1-3 h or longer if required. The temperature was then adjusted to 10-15° C. and a 23% NaHSO₃ solution (0.2 eq) was added dropwise at 10-20° C.; the mixture was stirred at 10-20° C. for 1-2 h. The mixture was filtered, the residue washed with 2-Me-THF (2.5-6.0 ml/g), and the filtrates were combined. The temperature was adjusted to 10-30° C., a 10% Na₂SO₃ solution (5 ml/g) was added dropwise, and the mixture stirred at 20-30° C. for 30-60 minutes. The mixture was allowed to stand at 20-30° C. for 30 to 60 minutes. The organic phase was separated and a 7% Na₂SO₄ solution (7 ml/g) was added. The mixture stirred at 20-30° C. for 20-40 minutes and was then allowed to stand at 20-30° C. for 1-2 h. The organic layer was separated and concentrated below 35° C. to 3-4 ml/g. 2-Me-THF was repeatedly added and the mixture concentrated below 35° C. to 3-4 ml/g until KF <0.2%. ¹H-NMR (300 MHz, DMSO-d6) of a stripped aliquot: δ 7.60 (m, 2H), 7.21 (m, 2H), 6.55 (t, 1H), 4.25 (d, 2H).

### Example 1-4: Preparation of (Z)-1-(3-bromo-1-iodoprop-1-en-1-yl)-4-fluorobenzene (Compound 4-4a)

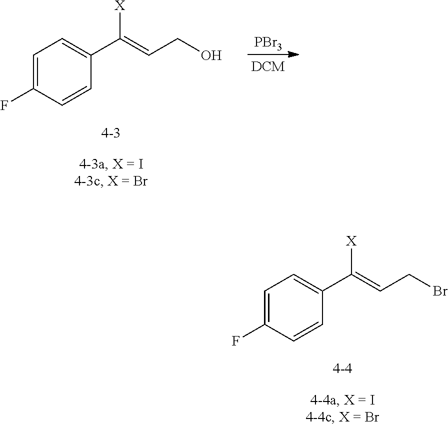

A solution of (Z)-3-(4-fluorophenyl)-3-iodoprop-2-en-1-ol (4-3a, 7.5 kg, 27 mol) in toluene (46 L) in a 100 L jacketed reactor was covered with black plastic sheet to protect the reaction solution from light. After cooled to 0° C., PBr₃ (973 mL, 10.5 mol) was added dropwise while keeping the reaction temperature below 5° C. After the addition, the resulting mixture was stirred for 60 minutes to reach a full conversion. A solution of 10% K₂HPO₄ (1.6 Kg K₂HPO₄·3H₂O in 17 L H₂O) was added and the mixture was allowed to stir for 30 minutes. The organic layer was siphoned out and the aqueous layer was extracted with ethyl acetate (5 L). The organic layers were combined and washed with 10% brine, dried with MgSO₄, and concentrated under reduced pressure to afford 7.83 Kg of the product. ¹H-NMR (300 MHz, CDCl₃): δ 7.50-7.44 (m, 2H), 7.07-7.02 (m, 2H), 6.19-6.14 (m, 1H), 4.22 (d, J=3.9, 2H).

### Example 1-5: Preparation of (Z)-1-(1,3-dibromoprop-1-en-1-yl)-4-fluorobenzene (Compound 4-4c)

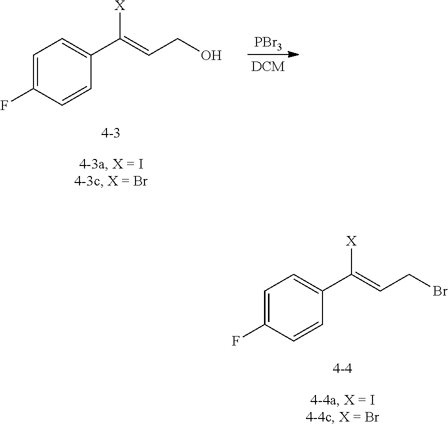

A solution of Compound 4-3c in 2-Me-THF was diluted with dichloromethane (DCM, 4 ml/g), and the mixture was concentrated to 2-5 ml/g, keeping the temperature below 35° C. This was repeated twice more, then the temperature of the mixture was adjusted to between −5 and 5° C. PBr₃ (0.4 eq) was added at between −5 and 5° C. and the reaction stirred at between −5 and 5° C. for 3-5 h. The reaction was monitored by IPC and further charges of PBr₃ (0.05 eq) were made as required. The reaction mixture continued stirring at between −5 and 5° C. for 3-5 h until IPC indicated reaction completion (<2% Compound 4-93c remained). n-heptane (10 ml/g) was added and the pH was adjusted to 3-5 with a 10% K₂HPO₄ solution (0.85 eq) at between −5 and 5° C. The mixture was warmed to 20-30° C. and was stirred at 20-30° C. for 20-40 minutes. The mixture was then allowed to stand at 20-30° C. for 20-40 minutes. The organic layer was separated and washed with a 5% Na₂SO₄ solution (0.30 eq) stirring at 20-30° C. for 20-40 minutes and was then allowed to stand at 20-30° C. for 20-40 minutes. The organic phase was separated and concentrated below 35° C. to 2-4 ml/g. Heptane (7 ml/g) was added and the mixture was filtered through silica gel. The residue was washed with heptane (4 ml/g), and the filtrates were combined then concentrated to 2-5 ml/g.

### Example 1-6: Preparation of methyl 2-(4-acetyl-2-methylphenoxy)acetate (Compound 4-6)

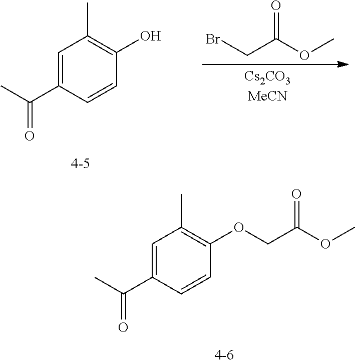

To a 100 L jacketed reactor was charged anhydrous acetonitrile (45 L) and 4-hydroxy-3-methylacetophenone (4-5, 4 kg, 26.6 mol). The mixture was cooled to 12° C. Cesium carbonate (13 kg, 40 mol) was added portionwise while keeping the temperature below 25° C. After the addition, the mixture was allowed to stir for 30 minutes and the temperature was lowered to 15° C. Methyl bromoacetate (2.6 L, 28 mol) was added while the temperature was maintained below 25° C. An exothermic reaction was observed. The reaction mixture was continued to stir at 25° C. overnight and the conversion was monitored by LC-MS. After the completion of the reaction, the mixture was filtered to remove inorganic salts and the filter cake was washed with acetonitrile (2×4 L). The filtrate and washing solutions were combined and concentrated under reduced pressure. The resulting solid was dissolved in ethyl acetate (20 L) and washed with H₂O (20 L). The mixture was allowed to stir for 30 minutes and then allowed to separate the layers. After removal of aqueous layer, the organic layer was dried with MgSO₄, filtered, and concentrated under reduced pressure to provide 6.0 kg of methyl 2-(4-acetyl-2-methylphenoxy)acetate. ¹H-NMR (300 MHz, CDCl₃): δ 7.80-7.76 (m, 2H), 6.73 (d, J=8.1 Hz, 1H), 4.73 (s, 2H), 3.81 (s, 3H), 2.55 (s, 3H), 2.32 (s, 3H).

### Alternative Conditions

A solution of Compound 4-5 in acetonitrile (11 ml/g) was cooled to 5-10° C. To the solution containing Compound 4-5 is added Cs₂CO₃ at 5-10° C. and the reaction mixture was stirred at 5-10° C. for 30-60 minutes. Next, to the reaction mixture was added methyl-2-bromoacetate (1.05 eq) at 5-10° C., stirring at 5-10° C. for 3-5 h or longer, until IPC indicated no more than 2% of Compound 4-5 was present in the reaction mixture. Additional charges of methyl-2-bromoacetate (0.05-0.1 eq) were added if necessary. The mixture was then filtered and concentrated below 35° C. to 2-4 ml/g. The mixture was repeatedly diluted with DCM and concentrated. Water was added and the mixture was stirred for 20-30 minutes at 20-25° C., and was then allowed to stand for 20-30 minutes at 20-25° C. The organic layer, containing a solution of Compound 4-6 in DCM, was separated and carried forward.

### Example 1-7: Preparation of methyl 2-(4-acetoxy-2-methylphenoxy)acetate (Compound 4-7)

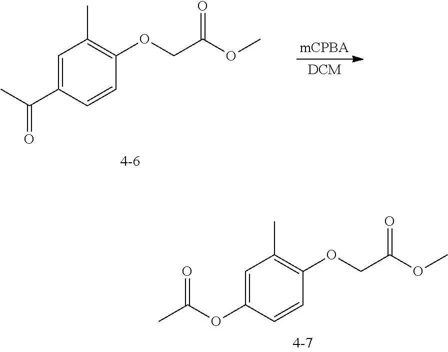 [SMILES: COC(=O)COc1ccc(C(C)=O)cc1C ; COC(=O)COc1ccc(OC(C)=O)cc1C]

To a 100 L jacketed reactor was charged compound 4-6 (6.4 kg, 28.8 mol), dichloromethane (50 L), and 85% m-CPBA (8.77 kg, 43.2 mol). The reaction temperature mixture was heated to reflux (40° C.) and stirred overnight. After completion of the reaction, the reaction mixture was cooled to room temperature. The reaction mixture was then treated with 1 M Na₂SO₃ (25 L), 2M Na₂CO₃ (25 L), saturated Na₂CO₃ (2×20 L), and brine (2×20 L). Each time the bi-phasic mixture was stirred for 10-15 minutes then allowed to separate the layers. The aqueous layer was separated; the organic layer was dried over anhydrous Na₂SO₄, filtered, and concentrated under reduced pressure to give 7.24 kg of the desired product. ¹H-NMR (300 MHz, CDCl₃): δ 6.91-6.83 (m, 2H), 6.71-6.68 (m, 1H), 4.64 (s, 2H), 3.81 (s, 3H), 2.29 (m, 6H).

### Alternative Conditions

To a reaction vessel containing a DCM solution of Compound 4-6 (1 eq, 0.1-5 ml/g) at 16-21° C. was added m-CPBA (0.5 eq), and the reaction was stirred at 16-21° C. for 20-30 minutes. Two additional charges of m-CPBA (0.5 eq) were made, stirring at 16-21° C. for 20-30 minutes. The temperature of the reaction mixture was adjusted to 19-24° C., and the reaction was stirred at 19-24° C. for 20-30 h or longer, until IPC indicated that the amount of Compound 4-6 was less than 3% of the amount of Compound 4-7. Upon completion, the reaction was quenched with 1M Na₂SO₃ solution (30 ml/g), maintaining a temperature of between 15-25° C. during the addition. The mixture was then stirred at 20-30° C. for 5-10 h before filtering and washing the residue with DCM. The organic layer was twice washed with 2M Na₂CO₃ solution, stirring at 15-25° C. for 30-60 minutes and then standing for 30-60 minutes before separating the organic phase. The organic solution containing Compound 4-7 was finally washed with water (3 ml/g) and concentrated to 3-5 ml/g below 45° C. The purity, assay and KF results of the product solution were determined.

### Example 1-8: Preparation of methyl 2-(4-hydroxy-2-methylphenoxy)acetate (Compound 4-8)

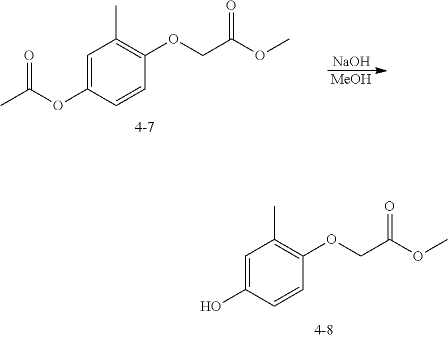

To a 100 L jacketed reactor was charged anhydrous methanol (48 L), compound 4-7 (6.9 kg, 28.9 mol), and sodium hydroxide (463 g, 11.57 mol). The reaction mixture was stirred at room temperature for 2 hrs. The progress of the reaction was followed by LC-MS. At nearly the complete conversion, the reaction was stopped. The solvent was removed under reduced pressure and the residue was dissolved in ethyl acetate (25 L). The organic solution was washed with water (20 L), saturated sodium bicarbonate (20 L), and brine (20 L). At each wash, the mixture was stirred for 10-15 minutes; the layers were then separated and the aqueous layer was removed. The organic layer was dried with Na₂SO₄, filtered, and concentrated under reduced pressure to afford 4.25 kg of the crude product as a light pink solid. The solid was re-dissolved in a minimal amount of ethyl acetate and crystallized by the addition of hexane at 60° C. 3.3 kg of the desired product was obtained. ¹H-NMR (300 MHz, CDCl₃): δ 6.67-6.64 (m, 1H), 6.61-6.57 (m, 2H), 4.76 (brs, 1H), 4.60 (s, 2H), 3.81 (s, 3H), 2.26 (s, 3H).

### Alternative Conditions

The solution of Compound 4-7 in DCM from the prior step was concentrated to 2-4 ml/g below 45° C. To the mixture was added MeOH (4-4.5 ml/g), and the mixture was concentrated to 3-5 ml/g below 45° C. The addition of MeOH and volume reduction was repeated twice more before adding Na₂CO₃ (0.40 eq). The mixture was adjusted to 15-25° C. and stirred at 15-25° C. for 5-10 h. Isopropyl acetate (4 ml/g) was added and the mixture was stirred at 15-25° C. for 5-10 minutes before filtering and concentrating to 4-5V below 45° C. Further isopropyl acetate was repeatedly added and the mixture repeatedly concentrated. Next, 10% Na₂SO₄ (3 ml/g) was added and the mixture was stirred at 20-30° C. for 15-30 minutes before being allowed to stand for 30-60 minutes. The organic layer was separated, again washed with 10% Na₂SO₄ (3 ml/g), stirring at 20-30° C. for 15-30 mins and then allowed to stand for 30-60 minutes. The organic phase was concentrated to 2-3V below 45° C. and adjusted to 55-65° C. Methylcyclohexane (5-18 ml/g) was added dropwise at 55-65° C. The mixture was cooled slowly to 15-25° C. and stirred at 15-25° C. for 1-12 h. The product was isolated by filtration, washed with methylcyclohexane (1-2 ml/g) and dried at 40-50° C. for 18-24 h. Purity, assay and KF data were generated.

### Example 1-9: Preparation of methyl (Z)-2-(4-((3-(4-fluorophenyl)-3-iodoallyl)oxy)-2-methylphenoxy)acetate (Compound 4a)

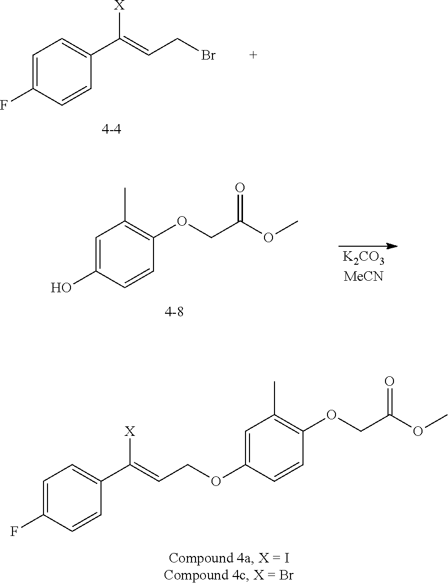

To a 100 L jacketed reactor were charged anhydrous acetonitrile (30 L), (Z)-1-(3-bromo-1-iodoprop-1-en-1-yl)-4-fluorobenzene (Compound 4-4a, 4.96 kg, 14.5 mol), and potassium carbonate (6.0 kg, 43.5 mol). The reactor was covered with a black plastic sheet to prevent the reaction solution from light. Methyl 2-(4-hydroxy-2-methylphenoxy)acetate (Compound 4-8, 3.0 kg, 15.3 mol) and cesium carbonate (950 g, 2.9 mol) were added to the mixture. The resulting mixture was stirred at rt for three days. Additional 20% of cesium carbonate (950 g, 2.9 mol) was added to push the reaction to completion. The reaction mixture was filtered through a pad of Celite. The filter cake was rinsed with acetonitrile (2×4 L). The organic solvent was removed and the resulting oil was re-dissolved in ethyl acetate (15 L). The organic solution was washed with brine (15 L), dried over Na₂SO₄, filtered, and concentrated under reduced pressure to give 6.2 kg of the crude product, which was dissolved with a minimal amount of toluene at 60° C. Hexanes was added and the mixture was allowed to crystallize. The resulting solid was filtered and washed with methanol to produce the desired product (Compound 4a) as white solid. ¹H-NMR (300 MHz, CDCl₃): δ 7.49-7.45 (m, 2H), 7.04-6.98 (m, 2H), 6.79 (s, 1H), 6.69 (d, J=1.2 Hz, 2H), 6.30 (t, J=5.1 Hz, 1H), 4.70 (d, J=4.8 Hz, 2H), 4.62 (s, 2H), 3.81 (s, 3H), 2.30 (s, 3H); LC-MS: m/z=479 (M+Na⁺).

### Example 1-10: Preparation of methyl (Z)-2-(4-((3-bromo-3-(4-fluorophenyl)allyl)oxy)-2-methylphenoxy)acetate (Compound 4c)

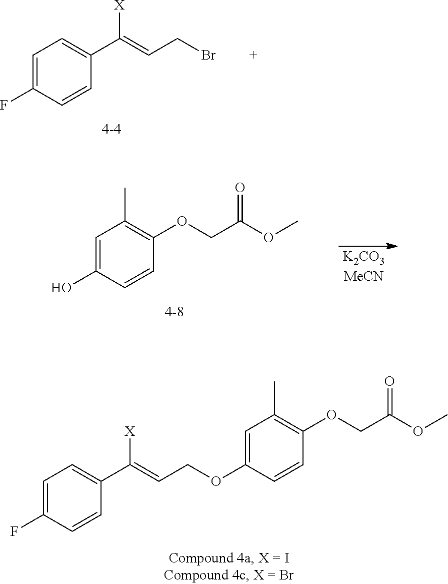

A heptane solution of Compound 4-4c (1.05 eq) was concentrated below 35° C. to 2-3 ml/g. Acetonitrile (4 ml/g) was added and the mixture was re-concentrated below 35° C. to 2-3 ml/g before additional acetonitrile (8 ml/g) was added. Methyl 2-(4-hydroxy-2-methylphenoxy)acetate (Compound 4-8), K₂CO₃ (2 eq) and Cs₂CO₃ (0.3 eq) were added to the reaction mixture, and the temperature of the reaction mixture was adjusted to 20-30° C. The mixture was stirred at 20-30° C. for 5-10 h or longer until IPC indicated <3% of Compound 4-8 remained. The mixture was filtered, the residue washed with ethyl acetate (1-2 ml/g) and the filtrates combined and concentrated below 45° C. to 2-4 ml/g. Ethyl acetate (6 ml/g) was added and the mixture concentrated below 45° C. to 6-8 ml/g. This procedure was repeated until acetonitrile levels were below 10% in the ethyl acetate solution. A 10% Na₂SO₄ solution (3 ml/g) was added and the mixture was stirred at 15-25° C. for 30-60 minutes. The mixture was then allowed to stand for 30-60 minutes. This procedure was repeated, and the organic layer was separated and concentrated below 45° C. to 2-3 ml/g. MeOH (6 ml/g) was added dropwise and the mixture was concentrated below 45° C. to 2-3 ml/g. This process was repeated until ethyl acetate levels were ≤10% in the distillate. Ethyl acetate was added (0.1-1 ml/g) to the mixture, which was then adjusted to 55-65° C. and then slowly cooled to 15-25° C. and stirred for 0.5-1 h. Compound 4c was filtered, washed with MeOH and dried at 30-50° C. for 18-24 h or until residual MeOH ≤1% and KF_≤1%.

### Example 2: Preparation of (E)-2-(4-((3-(4-fluorophenyl)-3-(4-(3-morpholinoprop-1-yn-1-yl)phenyl)allyl)oxy)-2-methylphenoxy)acetic Acid (Compound I)

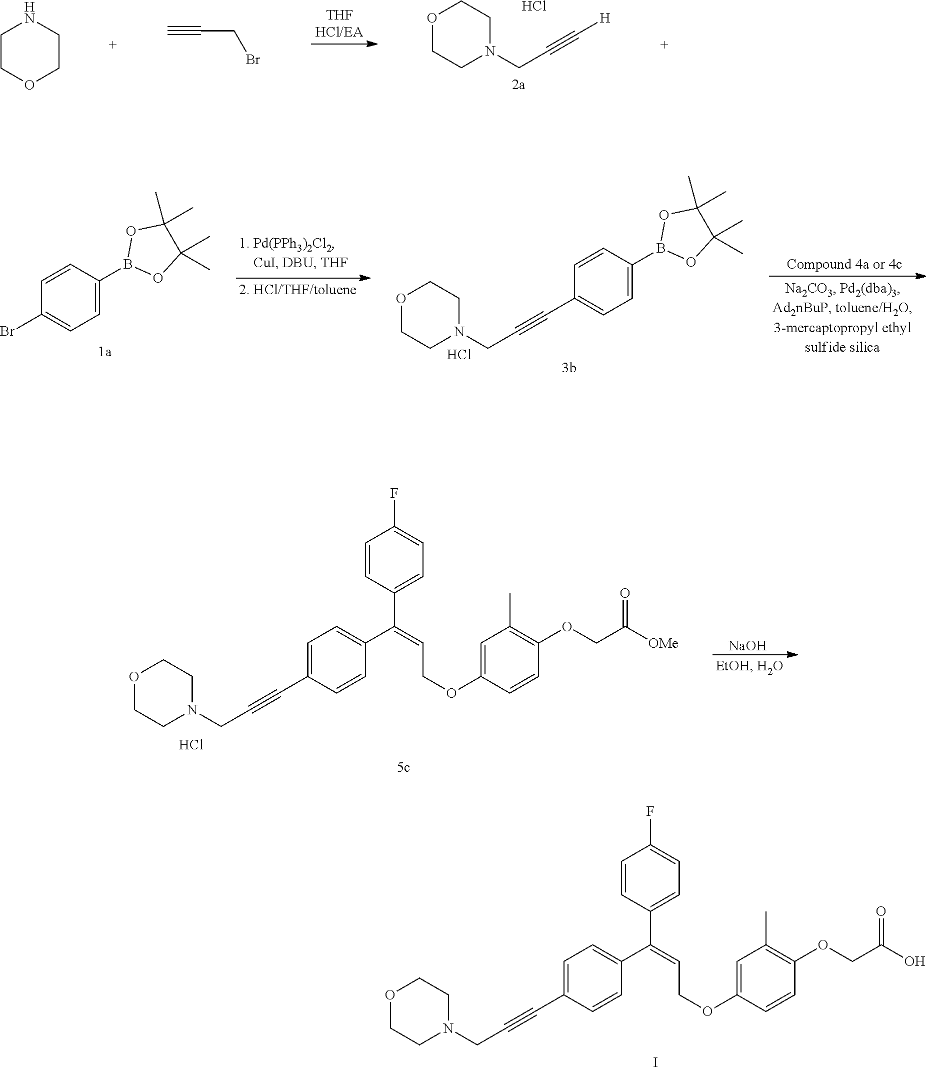

### Example 2-1: Preparation of 4-(prop-2-yn-1-yl)morpholine Hydrochloride (Compound 2a)

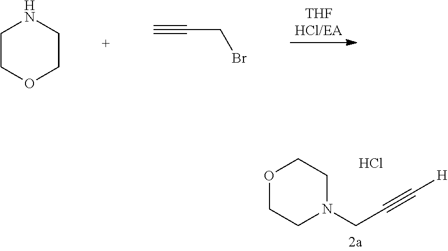

To a reaction vessel was added propargyl bromide (1 eq) and morpholine (1.95 eq) in THF (8 ml/g), keeping the temperature between 10-20° C. The temperature was adjusted to 15-25° C. and the mixture was stirred at 15-25° C. for 1-2 h, monitored by IPC. Additional charges of propargyl bromide or morpholine were made, if required, until and the mixture had stirred at 15-25° C. for 1-2 hrs. Upon completion of the reaction, the final mixture was filtered. A HCl/EA solution was prepared (2 M, 1.5 eq) and added to the filtrate, keeping the temperature between 10-20° C. The temperature was adjusted to 15-25° C. and the mixture was stirred at 15-25° C. for 2-5 h. The HCl gas in the reaction mixture was removed under reduced pressure for 1-3 h. The product was isolated by filtration, washed with THF, and vacuum dried at 20-30° C. for 3-6 h, followed by additional drying at 40-50° C. for 10-20 h. The product was sampled for KF IPC and further dried at 40-50° C. for 10-20 h if required. Purity was assessed by HPLC and KF.

### Example 2-2: Preparation of 4-(3-(4-(4,4,5,5-tetramethyl-1,3,2-dioxaborolan-2-yl)phenyl)prop-2-yn-1-yl)morpholine Hydrochloride (Compound 3b)

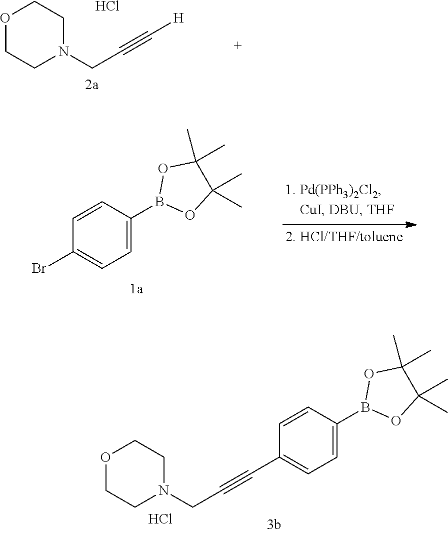 [SMILES: C#CCN1CCOCC1 ; Cl ; CC1(C)OB(c2ccc(Br)cc2)OC1(C)C ; CC1(C)OB(c2ccc(C#CCN3CCOCC3)cc2)OC1(C)C ; Cl]

A Sonogashira reaction was carried out using compound 1a (1 equiv), compound 2a (1.1 equiv), Pd(PPh₃)₂Cl₂ (1 mol %), CuI (0.5 mol %), DBU (2.5 equiv), in THF (7 ml/g). First, compound 1a and 2a were stirred at 20-30° C. in THF for 0.5 to 1 h. DBU was added dropwise at 20-30° C. and the vessel was purged with N₂. CuI and Pd(PPh₃)Cl₂ were added under N₂ and the mixture was adjusted to 58-63° C. and stirred for 5-8 h. GC-MS showed traces of unreacted 2, unreacted 1a and desired product. When GC-MS showed <5% of the starting material remained, the reaction was cooled to 25-35° C. and filtered. The filtrate was adjusted to 15-25° C. and AcOH (1-2 eq) was added at 15-25° C. until pH 6-7 was achieved. The mixture was concentrated below 45° C. to 2-3 ml/g. Toluene (10 ml/g) was added and the mixture was concentrated below 45° C. to 4-6 ml/g and the temperature was adjusted to 20-30° C. Water (5-6 ml/g) was added and the mixture stirred at 20-30° C. for 20-40 minutes and allowed to stand at 20-30° C. for 1-2 h. The organic phase was separated and additional water (5-6 ml/g) was added. The mixture was stirred at 25-35° C. for 20-40 minutes, filtered and allowed to stand at 25-35° C. for 20-40 mins.

To convert the free base 3a to the HCl salt 3b, the organic layer was separated and a 2N HCl/THF solution was added at 15-25° C. while stirring. The mixture was allowed to let stir an additional 2-5 h at 15-25° C. The product was then filtered, washed with toluene, and dried at 20-30° C. for 3-6 h, followed by further drying at 45-55° C. for 10-20 h or longer until KF <3%, residual THF <1%, and toluene <3%. ¹H-NMR (400 MHz, D2O): δ 7.67 (m, 2H), 7.48 (m, 2H), 4.23 (s, 2H), 3.70-4.10 (br, 4H), 3.25-3.60 (br, 4H), 1.18 (s, 12H).

### Example 2-3: Preparation of methyl (E)-2-(4-((3-(4-fluorophenyl)-3-(4-(3-morpholinoprop-1-yn-1-yl)phenyl)allyl)oxy)-2-methylphenoxy)acetate (Compound 5c) from Compound 4a

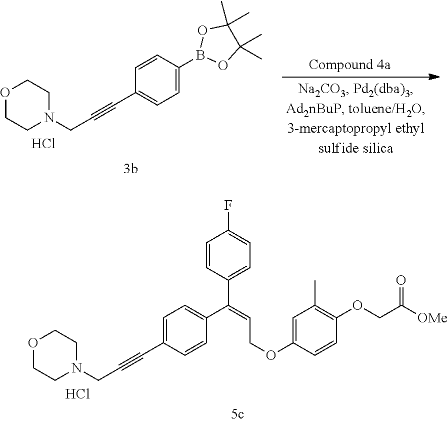 [SMILES: CC1(C)OB(c2ccc(C#CCN3CCOCC3)cc2)OC1(C)C ; Cl ; COC(=O)COc1ccc(OC/C=C(/c2ccc(F)cc2)c2ccc(C#CCN3CCOCC3)cc2)cc1C ; Cl]

A reaction vessel is charged with 5 g of Compound 3b (1 equiv), Compound 4a (1.1 equiv), Pd(PPh₃)₂Cl₂ (3 mol %), K₂CO₃ (3 equiv), MTBE:H₂O (1:1, 10 vol.). The reaction was heated at 60° C. for 48 h, then cooled to r.t., and the layers were separated. The organic phase was washed with 1M NaOH. The organic phase was further washed with water and brine.

### Removal of Residual Palladium

The organic phase was treated with 3-mercaptopropyl ethyl sulfide silica at 60° C. for 2 h, filtered, and the filtrates were reduced to ½ volume.

### Conversion to Compound 5c, the Hydrochloride Salt

2M HCl in ether was added, and the mixture was stirred for 2 h, filtered, and washed with MTBE to afford 5.8 g (74% yield) of Compound 5c. ¹H-NMR was consistent with structure.

### Example 2-4: Preparation of methyl (E)-2-(4-((3-(4-fluorophenyl)-3-(4-(3-morpholinoprop-1-yn-1-yl)phenyl)allyl)oxy)-2-methylphenoxy)acetate (Compound 5c) from Compound 4c

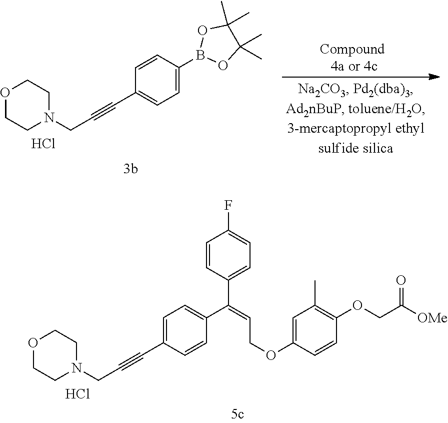 [SMILES: CC1(C)OB(c2ccc(C#CCN3CCOCC3)cc2)OC1(C)C ; Cl ; COC(=O)COc1ccc(OC/C=C(/c2ccc(F)cc2)c2ccc(C#CCN3CCOCC3)cc2)cc1C ; Cl]

A reaction vessel is charged with Compound 3b (1.1 eq) in MTBE (7 ml/g) and was stirred at 20-30° C. while a solution of Na₂CO3 (1.1 eq, 4-8 ml/g H₂O) was added. Next, Compound 4c was added to the mixture and the vessel was purged with N₂. Pd₂(dba)₃ (0.02 eq) and butyl di-1-adamantylphosphine (0.08 eq) were added under N₂ and the mixture was adjusted to 57-62° C. The reaction was stirred at 57-62° C. for 4-12 h. The reaction mixture was then diluted with MTBE (1-3 ml/g) and stirred at 57-62° C. for 4-12 h. This process was repeated, and the reaction monitored by IPC, stirring at 57-62° C., until less than 5% of the starting material remains. When IPC showed less than 5% of the starting material remained, the mixture was then cooled to 20-30° C. and adjusted to pH 5-7 with AcOH. The reaction mixture was filtered and allowed to stand at 20-30° C. for 30-60 minutes. The organic phase was then separated and a 5% citric acid solution (5-7 ml/g) was added. The mixture was stirred at 20-30° C. for 30-60 minutes and allowed to stand at 20-30° C. for 30-60 minutes. This process was repeated, and a final water wash carried out (5 ml/g), stirring at 20-30° C. for 30-60 minutes. The organic phase was separated and concentrated below 50° C. to 3-5 ml/g. Toluene (8 ml/g) was then added and concentrated below 50° C. to 4-5 ml/g. n-Heptane (3-6 ml/g) was then added and the mixture stirred at 20-30° C. for 3-6 h and filtered through diatomite.

### Removal of Residual Palladium

To the reaction mixture was added 3-mercaptopropyl ethyl sulfide silica, and the mixture was heated to 55-65° C. and stirred for 2-4 h before being filtered.

### Conversion of 5c to the Hydrochloride Salt

A 10% HCl/THF solution (1-2 ml/g) was added to the solution containing Compound 5c at 20-30° C. and was stirred for 1-3 h. The reaction temperature was then reduced to 0-10° C. and the solution was stirred for 2-5 h. The material was isolated by filtration, washed with toluene and dried at 40-50° C. for 10-20 h. The dry cake was mixed with water (10-15 ml/g) and stirred at 20-30° C. for 10-22 h. The mixture was filtered, washed with water and dried at 20-30° C. for 20-40 h to give Compound 5c purity was determined to be ≥95%, KF ≤5% (residual Pd ≤100 ppm and Cu ≤3000 ppm).

### Optional Purification of 5c

Compound 5c (5.07 kg) was triturated with acetonitrile (70.5 kg) at reflux (82° C.) for 10 minutes. The suspension was cooled to 22° C. and was filtered. The solid was washed with acetonitrile (9.0 kg) and was dried on the filter for 10 minutes. HPLC indicated 99.09 area % purity. Compound 5c was dried in a tray dryer at 43° C. under vacuum with a nitrogen purge for 22 h to yield 4.54 kg (70.9%) Compound 5c.

### Example 2-5: Preparation of (E)-2-(4-((3-(4-fluorophenyl)-3-(4-(3-morpholinoprop-1-yn-1-yl)phenyl)allyl)oxy)-2-methylphenoxy)acetic Acid (Compound I)

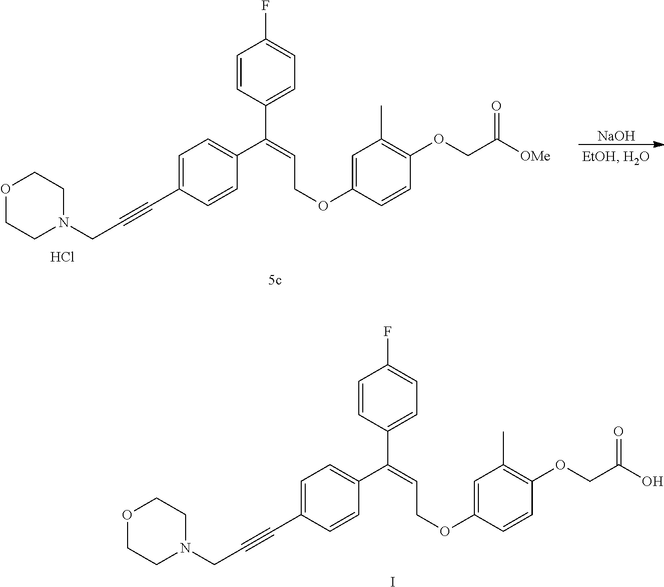

A reaction vessel is charged with Compound 5c, EtOH (6-10 ml/g), and water (2.5-4 ml/g) and was stirred at 15-25° C. A solution of aqueous NaOH (1.8 N, 2.5 eq) was added while the mixture was stirred, and the temperature was adjusted to 25-30° C., at which temperature the reaction continued to stir for 1-3 h. The reaction was monitored by IPC and stirring continued until Compound 5c/(Compound 5c+Compound I) was less than 1%. The mixture was then cooled to 15-25° C. The pH of the mixture was adjusted with a solution of AcOH (3.25 eq) in water (1-1.5 ml/g) and stirred at 15-25° C. for 2-3 h. The mixture was concentrated below 45° C. to 6-10 ml/g before water (4-6 ml/g) was added, facilitating isolation of Compound I by filtration. The filtrate was washed with water/EtOH 10:1. This washing was repeated until the purity of Compound I was no less than 98%. The product was dried at 45-55° C. for 10-20 hrs or longer until KF <3%.

In some instances, Compound I (3.99 kg) was triturated in 2-Me-THF (ACS grade, 36.2 kg) at 73-75° C. for 10 minutes. The suspension was cooled to 24° C. and filtered. The reactor was rinsed with 2-Me-THF (4.1 kg) and the rinse was sent to the filter. The solid was dried on the filter for 35 minutes and was further dried in a tray dryer under reduced pressure at 43° C. for 21 h to yield 3.34 kg (81.5% total yield) of Compound I as a white to off-white solid.

### Example 3: Alternative Preparation of (E)-2-(4-((3-(4-fluorophenyl)-3-(4-(3-morpholinoprop-1-yn-1-yl)phenyl)allyl)oxy)-2-methylphenoxy)acetic Acid (Compound I)

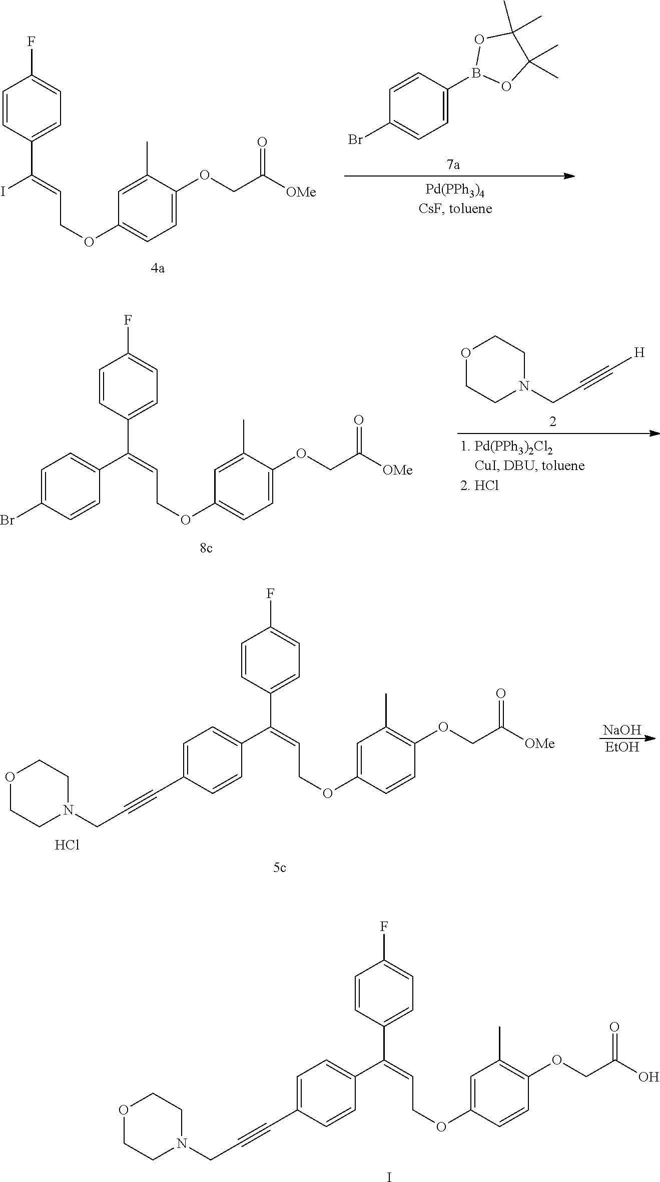

### Example 3-1: Preparation of methyl (Z)-2-(4-((3-(4-bromophenyl)-3-(4-fluorophenyl)allyl) oxy)-2-methylphenoxy)acetate (Compound 8c)

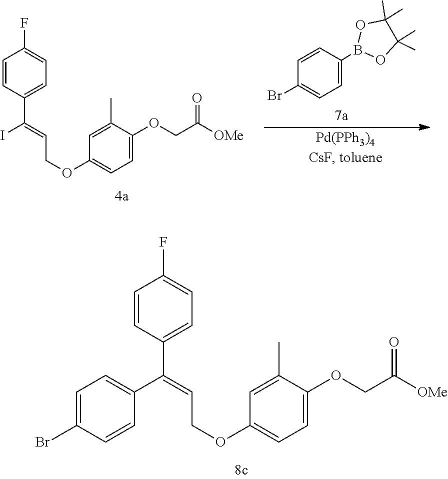 [SMILES: COC(=O)COc1ccc(OC/C=C(\I)c2ccc(F)cc2)cc1C ; CC1(C)OB(c2ccc(Br)cc2)OC1(C)C ; COC(=O)COc1ccc(OC/C=C(/c2ccc(F)cc2)c2ccc(Br)cc2)cc1C]

To a 72 L vessel was added 4a (3000 g, 6.575 mol), anhydrous toluene (35.5 L), boronate ester 7a (1334 g, 6.641 mol), and cesium fluoride (2000 g, 13.28 mol). The solution was degassed with nitrogen 45 min. Tetrakis(triphenylphosphine)palladium(0) (227.9 g, 0.1972 mol) was added and nitrogen was bubbled through the solution for 30 min. The reaction was stirred at 80° C. for 8 hr. HPLC analysis showed 6.5% 4a remaining. Additional 7a (13.3 g) was added and the reaction stirred 10 hr longer. HPLC analysis showed 3.3% 4a remaining. Additional 7a (13.3 g) was added and the reaction stirred 6 hr longer. HPLC analysis showed 2.3% 4a remaining. Additional 7a (13.3 g) was added and the reaction stirred 17 hr longer. HPLC analysis showed less than 1% 4a remaining. The reaction was cooled below 30° C., celite (2 kg) was added to the stirred solution, and the solution was filtered over celite (5 kg) in a 30 L glass filter. The celite pad was rinsed with toluene (8.5 L). The filtrates were poured into a clean 72 L vessel and the vessel was placed under nitrogen until the next step could be performed.

### Example 3-2: Preparation of methyl (E)-2-(4-((3-(4-fluorophenyl)-3-(4-(3-morpholinoprop-1-yn-1-yl)phenyl)allyl)oxy)-2-methylphenoxy)acetate (Compound 5c)

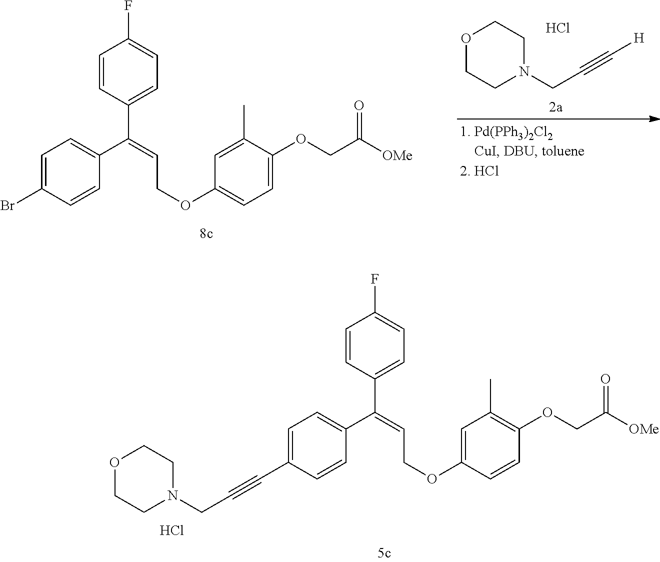 [SMILES: COC(=O)COc1ccc(OC/C=C(/c2ccc(F)cc2)c2ccc(Br)cc2)cc1C ; C#CCN1CCOCC1 ; Cl ; COC(=O)COc1ccc(OC/C=C(/c2ccc(F)cc2)c2ccc(C#CCN3CCOCC3)cc2)cc1C ; Cl]

To a 72 L vessel containing a toluene solution (44 L) of 8c (6.575 mol, assumed quantitative yield from the previous step) was added tetrahydrofuran (4.6 L), DBU (1301 ml, 8.547 mol) and 2a (987.6 g, 7.890 mol). The solution was degassed with nitrogen for 45 min. Copper(I) iodide (50.09 g, 0.2630 mol) was added to the solution. Nitrogen was bubbled through the solution for 10 min. Bis(triphenylphosphine)palladium(II) dichloride (187.4 g, 0.267 mol) was added and nitrogen was bubbled through the solution for 30 min. The reaction was stirred at 65° C. for 18 hr. The reaction was cooled below 30° C., celite (1 kg) and activated carbon (546.0 g) were added to the stirred solution, and the solution was filtered over celite (5 kg). The celite pad was rinsed with toluene (14 L) and the filtrates were poured into a clean 72 L vessel, which was cooled below 20° C. using an ice bath. Hydrochloric acid (808 ml) was added to adjust the pH of the solution below 4. The solution was cooled to 10° C., stirred 3 hr and was filtered. The filter cake was dried, rinsed with toluene (11 L) and was dried again. The filter cake was rinsed with water (5×10 L). The filter cake was dried on the filter for 19 hr and was further dried in a vacuum oven at 45° C. for 4 days to afford intermediate 5c (2968.2, 79.75% for 2 steps) as yellow-brown solid.

### Example 3-3: Removal of Residual Palladium from Compound 5c

To a 72 L vessel containing a solution of 5c (1481.5 g, 2.617 mol) in methanol (40 L) was added 3-mercaptopropyl ethyl sulfide silica (800.0 g). The solution was heated to 64.5° C. and stirred for 150 min under nitrogen. The solution was cooled to 50° C. and was filtered. The solids were washed with methanol (5.5 L). The filtrates were evaporated to 1/10 the original volume. The remaining methanol was azeotroped with toluene (3×3.33 L). Toluene (4.5 L) was added, the solution stirred on the rotovap at room temperature for 15 hrs and the solution was filtered. The filter cake was washed with toluene (4.5 L) and was air-dried on the filter 6 hrs. The solid was dried in a vacuum oven at 50° C. for 36 hrs to afford intermediate 5c as a beige solid. This reaction was run twice in this manner to yield: 1192.7 g (Sample #1, Palladium content=10 ppm, HPLC=99.13%) and 1255.0 g (Sample #2, Palladium content=13 ppm, HPLC=98.96%). Total=2447.7 g (82.6% recovery).

### Example 3-4: Preparation of (E)-2-(4-((3-(4-fluorophenyl)-3-(4-(3-morpholinoprop-1-yn-1-yl)phenyl)allyl)oxy)-2-methylphenoxy)acetic Acid (Compound I)

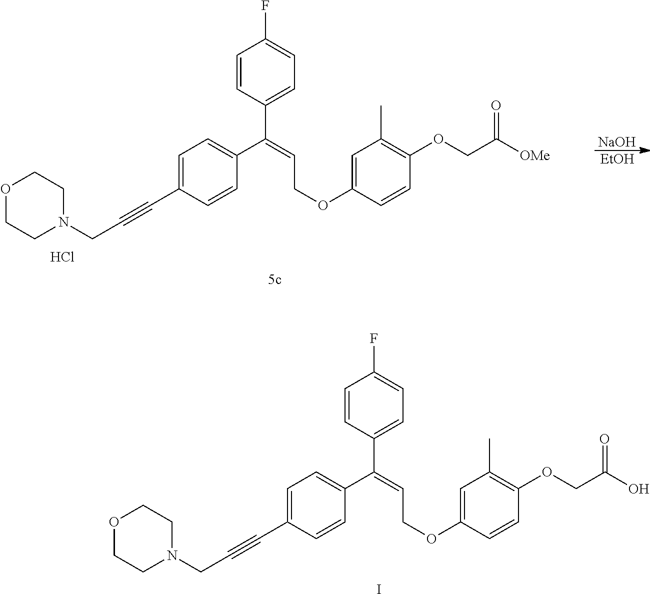

To a 72 L vessel containing a solution of intermediate 5c (1218.8 g, 2.153 mol) in ethanol (18 L) and water (6 L) was added a solution of sodium hydroxide (215.3 g, 5.383 mol) in water (3 L). The solution heated to 28.5° C. and stirred 3 hrs while cooling to 22.5° C. In a separate flask, acetic acid (400.0 ml) was dissolved in water (6.6 L). The entire (7 L) acetic acid solution was added to the 72 L vessel over 5 min to obtain pH 6. This mixture was stirred for 1 hr and then was concentrated under reduced pressure at 40° C. until all of the ethanol was removed (˜24 L of distillate). The remaining contents were transferred to another 72 L vessel and diluted with water (4.5 L). This mixture was stirred 1 hr and was filtered. The 72 L vessel was rinsed with water (2×5 L) and the rinse was transferred to the filter cake. The filter cake was air-dried 16 hrs and then in a vacuum oven at 50° C. for 50 hrs to afford compound I as light yellow solid. This reaction was run twice in this manner to yield: 1089.6 g (Sample #1, HPLC=99.7%) and 1099.2 g (Sample #2, HPLC=99.4%). Total=2188.8 g (98.6% yield).

### Example 4: Preparation of Sodium (E)-2-(4-((3-(4-fluorophenyl-3-(4-(3-morpholinoprop-1-yn-1-yl)phenyl)allyl)oxy)-2-methylphenoxy)acetate (Compound II)

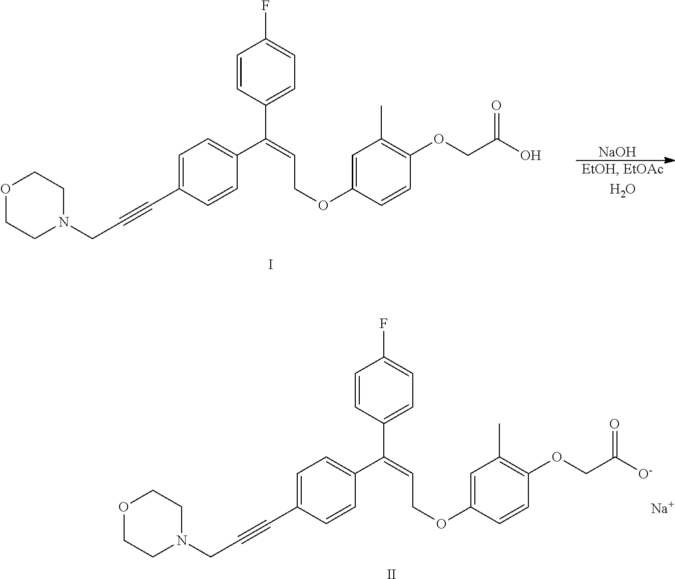

To a 72 L open head round bottom flask containing a solution of compound I (1089.4 g, 2.113 mol) in ethyl acetate (43 L) was added a solution of sodium hydroxide (82.0 g, 2.050 mol) in water (675 ml). The solution was heated to 40° C. and was filtered. The filtrates were concentrated under reduced pressure at 40° C. until 35 L of solvent were removed. The solution was stirred at 20° C. for 1 hr and was filtered. The filter cake was washed with ethyl acetate (4 L) and air-dried on the filter for 24 hrs followed by drying in a vacuum oven at 50° C. for 36 hrs to afford 1079.6 g of a beige solid. This solid was suspended in ethanol (22 L), was stirred 3 hrs at room temp and then was filtered. The filter cake was air-dried 2 hrs and then was slurried with ethanol (2×4 L) followed by filtration. The filter cake was air-dried 24 hrs and then transferred to a vacuum oven at 50° C. for 24 hrs to afford Compound II as a beige solid. This reaction was run twice in this manner to yield: 905.7 g (Sample #1, HPLC=99.85%, KF=0.65%, Acetic acid=19 ppm) and 968.7 g (Sample #2, HPLC=99.87%, KF=0.53%, Acetic acid=44 ppm). Total=1874.4 g (82.5% yield).

The two samples above were blended in a rotovap flask at room temperature for 1 hr to yield 1859.0 g of Compound II. ¹H-NMR (300 MHz, 1:1 CDCl₃/DMSO-d6): δ 7.45 (d, 2H), 7.22 (m, 2H), 7.15 (d, 2H), 7.04 (m, 2H), 6.65 (d, 1H), 6.59 (d, 1H), 6.50 (dd, 1H), 6.24 (t, 1H), 4.44 (d, 2H), 4.18 (s, 2H), 3.67 (m, 4H), 3.50 (s, 2H), 2.57 (m, 4H), 2.16 (s, 3H).

The examples and embodiments described herein are for illustrative purposes only and various modifications or changes suggested to persons skilled in the art are to be included within the spirit and purview of this application and scope of the appended claims.

What is claimed is:

1. A process for the preparation of Compound II: 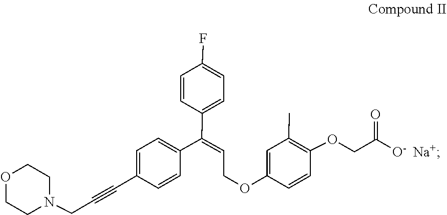 [SMILES: Cc1cc(OC/C=C(/c2ccc(F)cc2)c2ccc(C#CCN3CCOCC3)cc2)ccc1OCC(=O)[O-]]
comprising:
(1) reacting Compound 3, or a salt thereof: 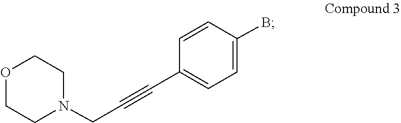
wherein B is a boronic acid, boronate ester, or trifluoroborate;
with Compound 4: 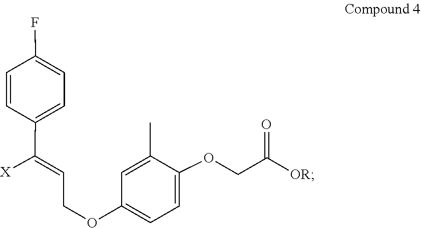
wherein R is C₁-C₆ alkyl; and
X is Br or I;
in the presence of a coupling catalyst, a suitable base, and in a suitable solvent, to provide Compound 5, or a salt thereof: 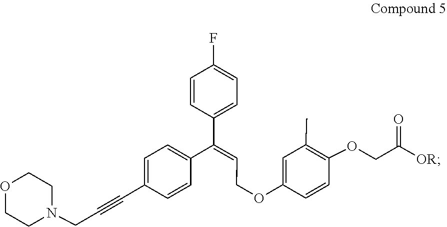
wherein R is C₁-C₆ alkyl;
(2) (i) reacting Compound 5 with sodium hydroxide, potassium hydroxide, or lithium hydroxide in a suitable solvent to provide Compound 6: 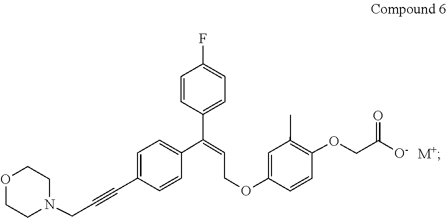
wherein M is sodium, potassium, or lithium;
and
(ii) contacting Compound 6 with a suitable acid in a suitable solvent to provide Compound I: 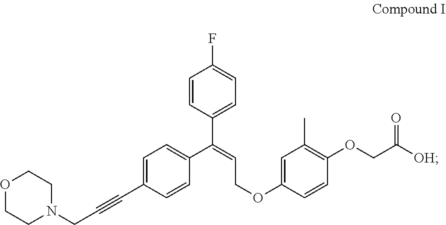 [SMILES: Cc1cc(OC/C=C(/c2ccc(F)cc2)c2ccc(C#CCN3CCOCC3)cc2)ccc1OCC(=O)O]
and
(3) reacting Compound I with a sodium hydroxide solution in the presence of a suitable solvent to provide Compound II.

2. The process of claim 1, wherein:
wherein B is a boronic acid or a boronate ester; and
R is methyl, ethyl, propyl, isopropyl, butyl, isobutyl, tert-butyl, isoamyl, pentyl, or hexyl.

3. The process of claim 1, wherein:
B is the boronic acid pinacol ester;
R is methyl, ethyl, propyl, isopropyl, butyl, isobutyl, tert-butyl, isoamyl, pentyl, or hexyl.

4. The process of claim 2 or 3, wherein:
R is methyl or ethyl; and
X is I.

5. The process of any one of claims 1-4, wherein Compound 4 is Compound 4a or Compound 4c:

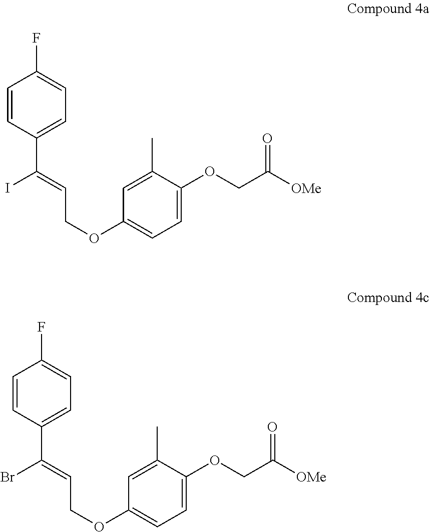 [SMILES: COC(=O)COc1ccc(OC/C=C(\I)c2ccc(F)cc2)cc1C ; COC(=O)COc1ccc(OC/C=C(\Br)c2ccc(F)cc2)cc1C]

6. The process of any one of claims 1-5, wherein the Compound 5 is:

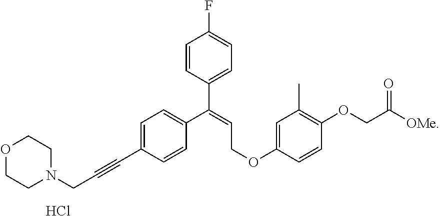 [SMILES: COC(=O)COc1ccc(OC/C=C(/c2ccc(F)cc2)c2ccc(C#CCN3CCOCC3)cc2)cc1C ; Cl]

7. The process of any one of claims 1-6, wherein:
the coupling catalyst of step (1) is a palladium catalyst;
the suitable base of step (1) is triethylamine, diisopropylethylamine, 1,2,2,6,6-pentamethylpiperidine, tributylamine, 1,8-diazabicyclo[5.4.0]undec-7-ene (DBU), sodium bicarbonate, Na₂CO₃, K₂CO₃, Cs₂CO₃, NaOAc, KOAc, Ba(OH)₂, Na₃PO₄, or K₃PO₄; and
the suitable solvent of step (1) is acetonitrile, dimethylformamide, dimethoxyethane, 2-methyltetrahydrofuran, methyl tert-butyl ether, cyclopentyl methyl ether, tetrahydrofuran, diisopropyl ether, 1,4-dioxane, toluene, water, or a combination thereof.

8. The process of claim 7, wherein:
the coupling catalyst of step (1) is a palladium catalyst;
the suitable base of step (1) is Na₂CO₃; and
the suitable solvent of step (1) is a mixture of toluene and water.

9. The process of claim 7 or 8, wherein:
step (1) is performed at a temperature of about 77-82° C.

10. The process of any one of claims 1-9, wherein:
the suitable solvent of step (2)(i) is water, methanol, ethanol, tetrahydrofuran, ethyl acetate, or a combination thereof.

11. The process of any claim 10, wherein:
the Compound 5 is reacted with sodium hydroxide in step (2)(i); and
the suitable solvent of step (2)(i) is a mixture of water and ethanol.

12. The process of claim 10 or 11, wherein:
step (2)(i) is performed at a temperature of about 15-25° C.

13. The process of any one of claims 1-12, wherein:
the suitable acid of step (2)(ii) is acetic acid, citric acid, oxalic acid, lactic acid, hydrochloric acid, nitric acid, or sulfuric acid; and
the suitable solvent of step (2)(ii) is water, methanol, ethanol, tetrahydrofuran, ethyl acetate, or a combination thereof.

14. The process of any claim 13, wherein:
the suitable acid of step (2)(ii) is acetic acid; and
the suitable solvent of step (3)(ii) is water.

15. The process of any one of claims 1-14, wherein:
Compound 6 is not isolated prior to step (2)(ii).

16. The process of any one of claims 1-15, wherein:
the suitable solvent of step (3) is water, methanol, ethanol, tetrahydrofuran, ethyl acetate, or a combination thereof.

17. The process of any one of claims 1-16, further comprising treatment of Compound 5 with a metal scavenger.

18. The process of claim 17, wherein:
the metal scavenger comprises SiO₂, charcoal, aqueous solution of L-cysteine, a Silicycle metal scavenger, Si-thiol, SiliaBond DMT, SiliaBond Cysteine, or 3-mercaptopropyl ethyl sulfide silica.

19. A process for the preparation of Compound 5, or a salt thereof: 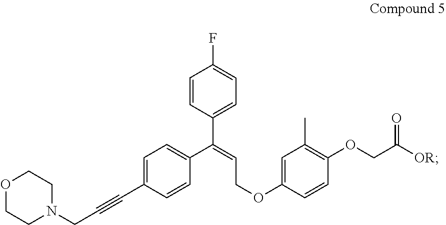
wherein R is C₁-C₆ alkyl;
comprising:
reacting Compound 3, or a salt thereof: 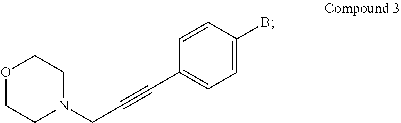
wherein B is a boronic acid, boronate ester, or trifluoroborate;
with Compound 4: 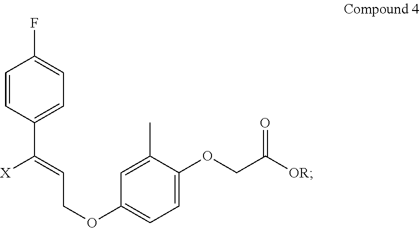
wherein R is C₁-C₆ alkyl; and
X is Br or I;
in the presence of a coupling catalyst, a suitable base, and in a suitable solvent, to provide Compound 5.

20. The process of claim 19, wherein:
B is a boronate ester; and
R is methyl, ethyl, propyl, isopropyl, butyl, isobutyl, tert-butyl, isoamyl, pentyl, or hexyl.

21. The process of claim 19, wherein:
B is the boronic acid pinacol ester;
R is methyl, ethyl, propyl, isopropyl, butyl, isobutyl, tert-butyl, isoamyl, pentyl, or hexyl.

22. The process of claim 19, wherein:
B is the boronic acid pinacol ester; and
R is methyl.

23. The process of any one of claims 19-22, wherein Compound 3, or salt thereof, is Compound 3b:

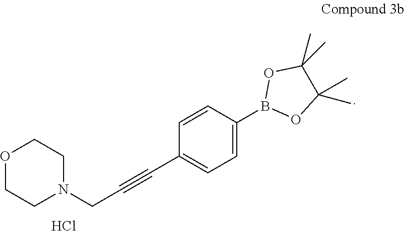 [SMILES: CC1(C)OB(c2ccc(C#CCN3CCOCC3)cc2)OC1(C)C ; Cl]

24. The process of any one of claims 19-23, wherein Compound 4 is Compound 4a:

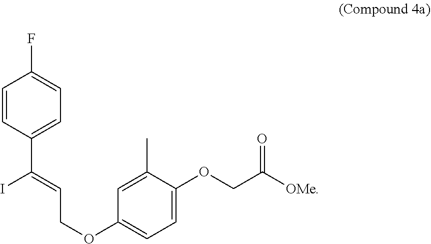 [SMILES: COC(=O)COc1ccc(OC/C=C(\I)c2ccc(F)cc2)cc1C]

25. The process of any one of claims 19-23, wherein Compound 4 is Compound 4c:

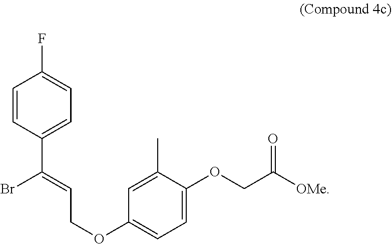 [SMILES: COC(=O)COc1ccc(OC/C=C(\Br)c2ccc(F)cc2)cc1C]

26. The process of any one of claims 19-25, wherein Compound 5 is:

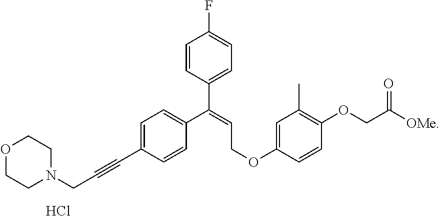 [SMILES: COC(=O)COc1ccc(OC/C=C(/c2ccc(F)cc2)c2ccc(C#CCN3CCOCC3)cc2)cc1C ; Cl]

27. The process of any one of claims 19-26, wherein:
the coupling catalyst is a palladium catalyst;
the suitable base is Na₂CO₃;
the suitable solvent is a mixture of toluene and water; and
the reaction is performed at a temperature of about 77-82° C.

28. The process of any one of claims 19-27, further comprising treatment of compound 5 with a metal scavenger.

29. The process of claim 28, wherein:
the metal scavenger comprises SiO₂, charcoal, aqueous solution of L-cysteine, a Silicycle metal scavenger, Si-thiol, SiliaBond DMT, SiliaBond Cysteine, or 3-mercaptopropyl ethyl sulfide silica.

30. The process of any one of claims 19-29, further comprising
(i) reacting Compound 5 with sodium hydroxide, potassium hydroxide, or lithium hydroxide in a suitable solvent to provide Compound 6: 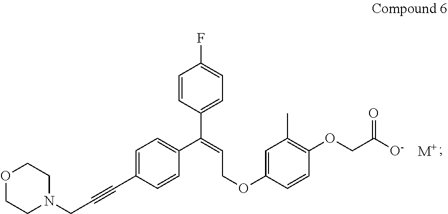
wherein M is sodium, potassium, or lithium
and
(ii) contacting Compound 6 with a suitable acid in a suitable solvent to provide Compound I:

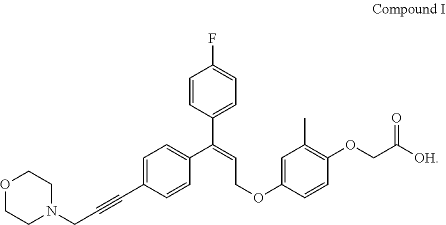 [SMILES: Cc1cc(OC/C=C(/c2ccc(F)cc2)c2ccc(C#CCN3CCOCC3)cc2)ccc1OCC(=O)O]

31. The process of claim 30, wherein:
Compound 5 is reacted with sodium hydroxide in step (i);
the suitable solvent of step (i) is a mixture of water and ethanol; and
step (i) is performed at a temperature of about 15-25° C.

32. The process of any one of claim 30 or 31, wherein:
the suitable acid of step (ii) is acetic acid; and
the suitable solvent of step (ii) is water.

33. The process of any one of claims 30-32, wherein:
Compound 6 is not isolated prior to step (ii).

34. The process of any one of claims 31-33, further comprising reacting Compound I with a sodium hydroxide solution in the presence of a suitable solvent to provide Compound II.

35. The process of claim 34, wherein:
the suitable solvent is a mixture of water, ethanol, and ethyl acetate.

36. The compound sodium (E)-2-(4-((3-(4-fluorophenyl)-3-(4-(3-morpholinoprop-1-yn-1-yl)phenyl)allyl)oxy)-2-methylphenoxy)acetate obtained by the process of any one of claims 1-18 or 30-35.

37. The compound methyl (E)-2-(4-((3-(4-fluorophenyl)-3-(4-(3-morpholinoprop-1-yn-1-yl)phenyl)allyl)oxy)-2-methylphenoxy)acetate, or a salt thereof, obtained by the process of any one of claims 19-29.

38. A process for the preparation of Compound 3, or salt thereof: 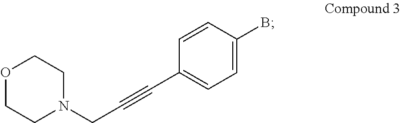
wherein B is a boronic acid, boronate ester, or trifluoroborate
comprising reacting Compound 1: 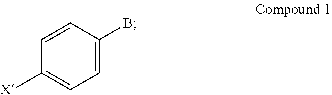
wherein X′ is Cl, Br or I;
with Compound 2, or salt thereof: 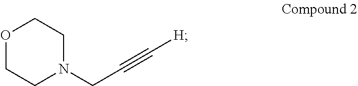
in the presence of a coupling catalyst, a suitable copper(I) cocatalyst, a suitable base, and in a suitable solvent.

39. The process of claim 38, wherein:
B is a boronate ester.

40. The process of claim 38, wherein:
B is the boronic acid pinacol ester.

41. The process of any one of claims 38-40, wherein:
X′ is Br.

42. The process of any one of claims 38-41, wherein Compound 1 is:

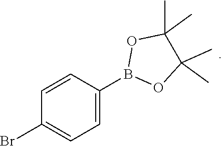 [SMILES: CC1(C)OB(c2ccc(Br)cc2)OC1(C)C]

43. The process of any one of claims 38-42, wherein:
the preparation of Compound 3, or salt thereof comprises reacting Compound 1 with Compound 2 or the hydrochloride salt of Compound 2:

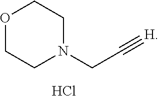

44. The process of any one of claims 38-43, wherein Compound 3, or salt thereof, is Compound 3b:

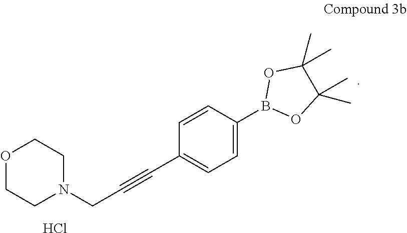 [SMILES: CC1(C)OB(c2ccc(C#CCN3CCOCC3)cc2)OC1(C)C ; Cl]

45. The process of any one of claims 38-44, wherein:
the coupling catalyst is a palladium catalyst;
the suitable copper(I) cocatalyst is CuI;
the suitable base is 1,8-diazabicyclo[5.4.0]undec-7-ene (DBU);
and the suitable solvent is tetrahydrofuran; and
the reaction is performed at a temperature of about 55-65° C.

46. The compound 4-(3-(4-(4,4,5,5-tetramethyl-1,3,2-dioxaborolan-2-yl)phenyl)prop-2-yn-1-yl)morpholine hydrochloride (Compound 3b) obtained by the process of any one of claims 38-45.

47. The compound 4-(3-(4-(4,4,5,5-tetramethyl-1,3,2-dioxaborolan-2-yl)phenyl)prop-2-yn-1-yl)morpholine hydrochloride (Compound 3b):

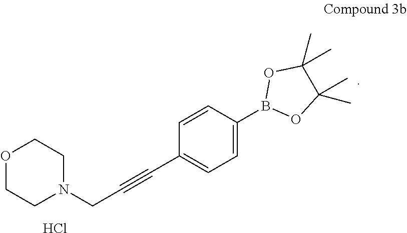 [SMILES: CC1(C)OB(c2ccc(C#CCN3CCOCC3)cc2)OC1(C)C ; Cl]

48. A compound having the following structure of Compound 4c:

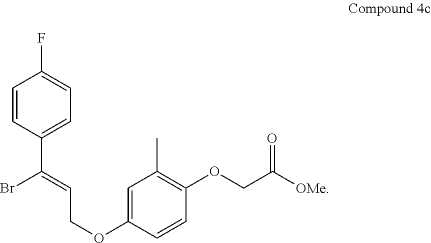 [SMILES: COC(=O)COc1ccc(OC/C=C(\Br)c2ccc(F)cc2)cc1C]

49. A process for the preparation of Compound 4: 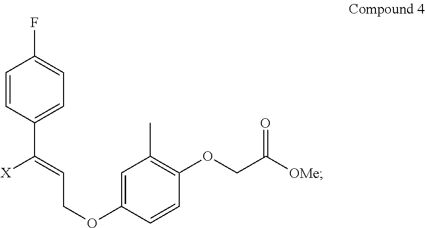
wherein X is Br or I;
comprising reacting Compound 4-8: 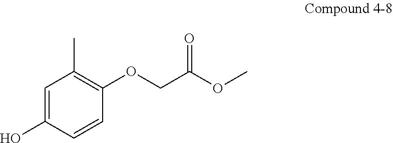
with Compound 4-4: 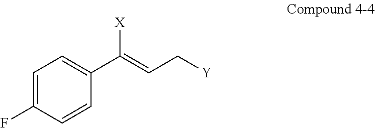
wherein X is Br or I; and Y is Br or Cl;
in the presence of a suitable base and in a suitable solvent to provide Compound 4.

50. The process of claim 49, wherein:
the suitable base is sodium bicarbonate, NaOAc, KOAc, Ba(OH)₂, Li₂CO₃, Na₂CO₃, K₂CO₃, Cs₂CO₃, Na₃PO₄, K₃PO₄, CsF, or combination thereof; and
the suitable solvent is acetonitrile, dimethylformamide, dimethoxyethane, 2-methyltetrahydrofuran, methyl tert-butyl ether, cyclopentyl methyl ether, tetrahydrofuran, diisopropyl ether, 1,4-dioxane, toluene, or a combination thereof.

51. The process of claim 50, wherein:
the suitable base is K₂CO₃, Cs₂CO₃, or combination thereof; and
the suitable solvent is acetonitrile.

52. The process of claim 49, wherein Compound 4-4 is prepared by a process comprising:
(1) reacting 4-fluoroiodobenzene or 4-fluorobromobenzene with propargyl alcohol: 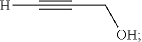
in the presence of a coupling catalyst, a suitable base, and in a suitable solvent, to provide Compound 4-2: 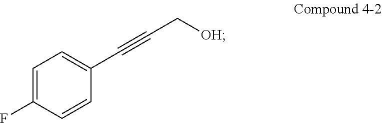 [SMILES: OCC#Cc1ccc(F)cc1]
(2) reacting compound 4-2 under suitable bromination conditions or suitable iodination conditions to provide Compound 4-3: 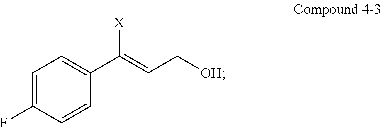
wherein X is Br or I;
(3) brominating Compound 4-3 with a suitable brominating agent in a suitable solvent to provide Compound 4-4, wherein Y is Br; or chlorinating Compound 4-3 with a suitable chlorinating agent in a suitable solvent to provide Compound 4-4, wherein Y is Cl.

53. The process of claim 52, wherein:
the coupling catalyst of step (1) is a palladium catalyst;
the suitable base of step (1) is triethylamine, diisopropylethylamine, 1,2,2,6,6-pentamethylpiperidine, tributylamine, or 1,8-diazabicyclo[5.4.0]undec-7-ene (DBU); and
the suitable solvent of step (1) is acetonitrile, dimethylformamide, diethyl ether, ethanol, 2-methyltetrahydrofuran, tetrahydrofuran, isopropyl alcohol, 1,4-dioxane, toluene, water, or a combination thereof.

54. The process of claim 53, wherein:
the coupling catalyst of step (1) is a palladium catalyst;
the suitable base of step (1) is diisopropylethylamine;
and the suitable solvent of step (1) is 2-methyltetrahydrofuran.

55. The process of any one of claims 52-54, wherein:
step (1) further comprises the use of a suitable Cu(I) cocatalyst.

56. The process of claim 55, wherein:
the suitable copper(I) cocatalyst is CuCl, CuBr, or CuI.

57. The process of claim 52, wherein:
the bromination of step (2) proceeds through:
(i) hydrometalation; and (ii) reaction with a bromonium (Br⁺) source, in a suitable solvent.

58. The process of claim 57, wherein:
hydrometalation in step (i) is performed by a metal hydride.

59. The process of claim 58, wherein:
the metal hydride is lithium aluminum hydride.

60. The process of claim 57, wherein:
the bromonium (Br⁺) source in step (ii) is N-bromo-succinimide (NBS).

61. The process of any one of claims 57-60, wherein:
the suitable solvent is dimethoxyethane, 2-methyltetrahydrofuran, methyl tert-butyl ether, cyclopentyl methyl ether, tetrahydrofuran, diisopropyl ether, 1,4-dioxane, or a combination thereof.

62. The process of any one of claims 57-61, wherein:
the suitable solvent is 2-methyltetrahydrofuran.

63. The process of claim 52, wherein:
the iodation of step (2) proceeds through:
(i) hydrometalation; and (ii) reaction with an iodonium (I⁺) source, in a suitable solvent.

64. The process of claim 63, wherein:
hydrometalation in step (i) is performed by a metal hydride.

65. The process of claim 64, wherein:
the metal hydride is lithium aluminum hydride.

66. The process of claim 63, wherein:
the iodonium (I⁺) source in step (ii) is iodine (I₂) or N-iodosuccinimide (NIS).

67. The process of any one of claims 63-66, wherein:
the suitable solvent is dimethoxyethane, 2-methyltetrahydrofuran, methyl tert-butyl ether, cyclopentyl methyl ether, tetrahydrofuran, diisopropyl ether, 1,4-dioxane, or a combination thereof.

68. The process of any one of claims 63-67, wherein:
the suitable solvent is tetrahydrofuran.

69. The process of any one of claims 52-68, wherein:
the suitable brominating agent in step (3) is PBr₃; and
the suitable solvent in step (3) is dichloromethane.

70. The process of claim 49, wherein Compound 4-8 is prepared by a process comprising:
(1) reacting Compound 4-5: 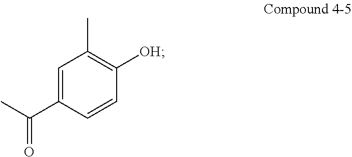
with methyl 2-bromoacetate: 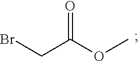
in the presence of a suitable base and in a suitable solvent, to provide compound 4-6: 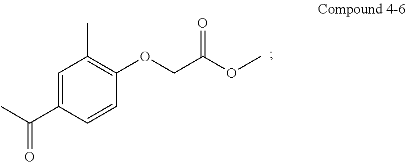 [SMILES: COC(=O)COc1ccc(C(C)=O)cc1C]
(2) reacting Compound 4-6 with a suitable oxidant and in a suitable solvent to provide Compound 4-7: 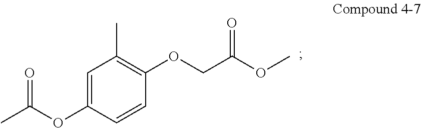 [SMILES: COC(=O)COc1ccc(OC(C)=O)cc1C]
(3) reacting compound 4-7 with a suitable base and in a suitable solvent to provide Compound 4-8.

71. The process of claim 70, wherein:
the suitable base in step (1) is sodium bicarbonate, NaOAc, KOAc, Ba(OH)₂, Li₂CO₃, Na₂CO₃, K₂CO₃, Cs₂CO₃, Na₃PO₄, K₃PO₄, or CsF; and
the suitable solvent in step (4) is acetonitrile, dimethylformamide, dimethoxyethane, 2-methyltetrahydrofuran, methyl tert-butyl ether, cyclopentyl methyl ether, tetrahydrofuran, diisopropyl ether, 1,4-dioxane, toluene, or a combination thereof.

72. The process of claim 71, wherein:
the suitable base in step (1) is Cs₂CO₃; and
the suitable solvent in step (1) is acetonitrile.

73. The process of any one of claims 70-72, wherein:
the suitable oxidant in step (2) is meta-chloroperbenzoic acid, peracetic acid, trifluoroperacetic acid, oxone, or hydrogen peroxide; and
the suitable solvent in step (2) is trifluoroacetic acid, dichloromethane, acetonitrile, dimethylformamide, dimethoxyethane, ethyl acetate, methanol, water, toluene, or a combination thereof.

74. The process of claim 73, wherein:
the suitable oxidant in step (2) is meta-chloroperbenzoic acid; and
the suitable solvent in step (2) is dichloromethane.

75. The process of any one of claims 70-74, wherein:
the suitable base in step (3) is NaOAc, KOAc, Li₂CO₃, Na₂CO₃, K₂CO₃, or Cs₂CO₃; and
the suitable solvent in step (3) is acetonitrile, methanol, ethanol, tetrahydrofuran, isopropyl alcohol, 1,4-dioxane, toluene, water, or a combination thereof.

76. The process of claim 75, wherein:
the suitable base in step (3) is Na₂CO₃; and
the suitable solvent in step (3) is acetonitrile or methanol.
